SEQUENCE LISTING (8 sequences)

SEQ ID NO: 1 - DNA, 26 bp, Artificial Sequence
  Description of Artificial Sequence Synthetic Primer
agtttctagaatggtgagcaagggcg

SEQ ID NO: 2 - DNA, 32 bp, Artificial Sequence
  Description of Artificial Sequence Synthetic Primer
atcgctcgagttagcactgcttgtcggccatg

SEQ ID NO: 3 - DNA, 21 bp, Artificial Sequence
  Description of Artificial Sequence Synthetic Primer
cgactgcgttagcatgtgttg

SEQ ID NO: 4 - DNA, 34 bp, Artificial Sequence
  Description of Artificial Sequence Synthetic Primer
atcggatatcatgtgcaagaacggcatcaaggtg

SEQ ID NO: 5 - DNA, 29 bp, Artificial Sequence
  Description of Artificial Sequence Synthetic Primer
atcgctcgagttacttgtacagctcgtcc

SEQ ID NO: 6 - DNA, 21 bp, Artificial Sequence
  Description of Artificial Sequence Synthetic Primer
caacacatgctaacgcagtcg

SEQ ID NO: 7 - protein, 238 aa, Aequorea victoria
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 8 - DNA, 966 bp, Aequorea victoria
tacacacgaataaaagataacaaagatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataaatgtccagacttccaattgacactaaagtgtccgaacaattactaaaatctcagggttcctggttaaattcaggctgagatattatttatatatttatagattcattaaaattgtatgaataatttattgatgttattgatagaggttattttcttattaaacaggctacttggagtgtattcttaattctatattaattacaatttgatttgacttgctcaaa

(19) United States
(12) Patent Application Publication
(10) Pub. No.: US 2009/0220942 A1
(43) Pub. Date: Sep. 3, 2009

(54) ACTIVATED SPLIT-POLYPEPTIDES AND METHODS FOR THEIR PRODUCTION AND USE

(76) Inventors: Natalia Broude, Natick, MA (US); Charles R. Cantor, Del Mar, CA (US); Vadim V. Demidov, Boston, MA (US)

Correspondence Address: RONALD I. EISENSTEIN, 100 SUMMER STREET, NIXON PEABODY LLP, BOSTON, MA, 02110, US

(21) Appl. No.: 12/091,709

(22) PCT Filed: Oct. 27, 2006
(86) PCT No.: PCT/US06/42299

§ 371 Date: Oct. 1, 2008

Related U.S. Application Data
(60) Provisional application No. 60/730,752, filed on Oct. 27, 2005.

(51) Int. Cl. C12Q 1/70; C12Q 1/68; C07K 14/00; C12P 21/00

(52) U.S. Cl. 435/5; 435/6; 530/350; 435/69.1; 435/68.1

(57) ABSTRACT

The present invention relates to a method to produce activated split-polypeptide fragments that on reconstitution immediately forms an active protein. The method relate to real-time protein complementation. Also encompassed in the invention is a method to split and produce split-fluorescent proteins in an active state which produce a fluorescent signal immediately on reconstitution. The present application also provides methods to detect nucleic acids; non-nucleic acid analytes and nucleic acid hybridization in real-time using the novel activated split-polypeptide fragments of the invention.

### CROSS REFERENCE TO RELATED APPLICATIONS

This application claims the benefit under 35 U.S.C. 119(e) of U.S. Provisional Patent Application Ser. No. 60/730,752, filed Oct. 27, 2005, the contents of which are herein incorporated by reference in their entirety.

### FIELD

The present invention provides novel activated split-polypeptide proteins for fast biomolecular protein complementation and methods for their production and their use.

### BACKGROUND

Protein complementation is a comparatively new method whereby a protein is split into two or more inactive fragments which can to reassemble for form an active protein. One limitation of use of inactive split-polypeptide fragments is that on reconstitution, they need to refold and reassemble in order to form the active protein. These poor folding characteristics limit the use of inactive split-polypeptides in protein complementation in methods to detect biomolecular interactions in real-time with fast kinetics.

GFP and its numerous related fluorescent proteins are now in widespread use as protein tagging agents (for review, see Verkhusha et al., 2003, Ch. 18, pp. 405-439). In addition, GFP has been used as a solubility reporter of terminally fused test proteins (Waldo et al., 1999, Nat. Biotechnol. 17:691-695; U.S. Pat. No. 6,448,087). GFP-like proteins are an expanding family of homologous, 25-30 kDa polypeptides sharing a conserved 11 beta-strand “barrel” structure. The GFP-like protein family currently comprises some 100 members, cloned from various Anthozoa and Hydrozoa species, and includes red, yellow and green fluorescent proteins and a variety of non-fluorescent chromoproteins (Verkhusha et al., supra). A wide variety of fluorescent protein labeling assays and kits are commercially available, encompassing a broad spectrum of GFP spectral variants and GFP-like fluorescent proteins, including DsRed and other red fluorescent proteins (Clontech, Palo Alto, Calif.; Amersham, Piscataway, N.J.).

Various strategies for improving the solubility of GFP and related proteins have been documented, and have resulted in the generation of numerous mutants having improved folding, solubility and perturbation tolerance characteristics. Existing protein tagging and detection platforms are powerful but have drawbacks. Split protein tags can perturb protein solubility (Ullmann, Jacob et al. 1967; Nixon and Benkovic 2000; Fox, Kapust et al. 2001; Wigley, Stidham et al. 2001; Wehrman, Kleaveland et al. 2002) or may not work in living cells (Richards and Vithayathil 1959; Kim and Raines 1993; Kelemen, Klink et al. 1999). Green fluorescent protein fusions can misfold (Waldo, Standish et al. 1999) or exhibit altered processing (Bertens, Heijne et al. 2003). Fluorogenic biarsenical FLaSH or ReASH (Adams, Campbell et al. 2002) substrates overcome many of these limitations, but require a polycysteine tag motif, a reducing environment, and cell transfection or permeabilization (Adams, Campbell et al. 2002).

GFP fragment reconstitution systems have been described, mainly for detecting protein-protein interactions, but none are capable of unassisted self-assembly into a correctly-folded, soluble and fluorescent re-constituted GFP. In addition, no general split GFP folding reporter system has emerged from these approaches. For example, Ghosh et al, 2000, reported that two GFP fragments, corresponding to amino acids 1-157 and 158-238 of the GFP structure, could be reconstituted to yield a fluorescent product, in vitro or by coexpression in E. coli, when the individual fragments were fused to coiled-coil sequences capable of forming an antiparallel leucine zipper (Ghosh et al., 2000, J. Am. Chem. Soc. 122: 5658-5659). Likewise, U.S. Pat. No. 6,780,599 describes the use of helical coils capable of forming anti-parallel leucine zippers to join split fragments of the GFP molecule. However, this method takes two days to acquire a positive signal and is thus too impractical for use.

Similarly, Hu et al., 2002, showed that the interacting proteins bZIP and Rel, when fused to two fragments of GFP, can mediate GFP reconstitution by their interaction (Hu et al., 2002, Mol. Cell. 9: 789-798). Nagai et al., 2001, showed that fragments of yellow fluorescent protein (YFP) fused to calmodulin and M13 could mediate the reconstitution of YFP in the presence of calcium (Nagai et al., 2001, Proc. Natl. Acad. Sci. USA 98: 3197-3202). In a variation of this approach, Ozawa at al. fused calmodulin and M13 to two GFP fragments via self-splicing intein polypeptide sequences, thereby mediating the covalent reconstitution of the GFP fragments in the presence of calcium (Ozawa et al., 2001, Anal. Chem. 72: 5151-5157; Ozawa et al., 2002 Anal. Chem. 73: 5866-5874).

Although the aforementioned GFP reconstitution systems provide advantages over the use of two spectrally distinct fluorescent protein tags, they are limited by the size of the fragments and correspondingly poor folding characteristics (Ghosh et al., Hu et al., supra), the requirement for a chemical ligation, and co-expression or co-refolding to produce detectable folded and fluorescent GFP (Ghosh et al., 2000; Hu et al., 2001, supra).

The poor folding characteristics limit the use of these fragments and other inactive split-polypeptide fragments because they have reduced fluorescence or take too long to fluoresce in vivo to be useful in real time assays. In addition, such fragments are not useful for in vitro assays requiring the long-term stability and solubility of the respective fragments prior to complementation.

The production of split-fluorescence polypeptides that do not need to be refolded on reconstitution for formation of the active protein would eliminate the lag time for the generation of an active protein, and could be used for real-time protein complementation assays.

An ideal split-polypeptide fragment would be genetically encoded, could work both in vivo and in vitro, provide a sensitive analytical signal that is reversible, and immediately produces an active protein and thus a signal upon target recognition. However, to date, already activated, split-polypeptide fragments that efficiently accomplishes the goal of real-time protein complementation has not been described.

### SUMMARY OF THE INVENTION

The present invention is directed towards a novel system for real time detection of target nucleic acid molecules, including DNA, RNA targets, as well as nucleic acid analogues and non-nucleic acid analytes. In particular, the invention comprises a molecule and methods for its production and use. The molecule of the invention can i) detects nucleic acids and non-nucleic acid analytes via reconstitution of activated split-polypeptides in real time and with little to no lag time between recognition and detection; and ii) reversibly increases and decreases its signal in response to detection of its target molecule, such as a nucleic acid or analyte. In one embodiment, the molecule is based on a hybridization-driven complementation of activated split-polypeptide fragments that form an active protein immediately on reconstitution. In another embodiment, the molecule is based on binding of a split-polypeptide fragment to a target analyte. Proteins used for protein complementation methods can be any protein that can be split into fragments and can reconstitute to form an active protein, in particular marker proteins that generate active proteins with enzymatic activity of fluorescent properties, for example fluorogenic activity or chromogenic activity. In one embodiment, the split-polypeptide is a fluorescent protein or polypeptide, where one of the split-fluorescent fragments contains preformed chromophores. In such an embodiment, as the chromophores is already formed and in its mature conformation, one does not need to wait until for chromophore formation for a fluorescent signal.

The molecule of the invention is useful for real-time monitoring of various biomolecular applications, such as nucleic acid diagnostics, pathogen monitoring and biocomputing.

The activated split-polypeptide of the current invention encompasses any polypeptide that can be split and on reassociation immediately forms an active protein. Such activated split-polypeptide comprise, for example proteins with enzymatic activity or fluorogenic activity, such as enzymes with chromogenic activity or fluorescent proteins.

One aspect of the present invention encompasses the production of the activated fluorescent polypeptide fragments containing a mature preformed chromophore which is capable of immediate fluorescence when associated with its corresponding fluorescent polypeptide partner, but is non-fluorescent when disassociated. In one embodiment, the chromophore is not fluorescent in the fragment because it is exposed to and quenched by solvent, and lacks necessary contacts with amino acids of the other fragment. When the two protein fragments are brought close to each other by nucleic acid complementary interactions, the second polypeptide acts as a shield for the chromophore isolating it from solution and allowing restoration of all missing amino acid contacts which results in immediate development of fluorescence. The presence of a preformed chromophore in one of the fragments allows for virtual immediate fluorescence upon association with its complementary protein fragment. Immediate fluorescence occurs because the chromophore is already formed, thus eliminating lag time required for its correct folding and formation.

In one embodiment, the invention provides novel methods for producing a split-polypeptide molecule, which can also be referred to as a biomolecular construct herein. The method provides for the in vitro isolation of activated split-polypeptide fragments, such as split fluorescent proteins where the chromophores is already present in one fragment. In particular, the split-polypeptide fragments are expressed in E. Coli as fusion proteins with small self-splitting Ssp DNAB intein. These polypeptides are isolated from inclusion bodies after refolding, which allows for the maturation, for example, of the chromophore within one fragment, but not its fluorescence. It is possible to purify inclusion bodies containing activated split-polypeptide proteins in a highly effective manner from host cell polypeptides and other host cell-derived impurities, as most of all substances contained in the inclusion bodies are easily soluble under denaturing conditions that allow for protein purification, but which do not denature the proteins. Intein facilitates protein purification and does not alter the structure of the split-polypeptide protein fragments. Peptides other than intein are known to those of skill in the art and can be used in the purification methods of the present invention.

In some embodiments, where the split-polypeptide fragment is a split-fluorescent protein, one fragment contains a mature preformed chromophore that is active but in a non-fluorescent state. The isolation of the chromophore in its mature, yet inactive, state allows for the ability to immediately detect fluorescence upon complementation with its corresponding fragment.

In one embodiment, the fluorescent protein is green fluorescent protein (GFP) or enhanced green fluorescent protein (EGFP). In alternative embodiments, the fluorescent protein is yellow fluorescent protein (YFP), an enhanced yellow fluorescent protein (EYFP), a blue fluorescent protein (BFP), an enhanced blue fluorescent protein (EBFP), a cyan fluorescent protein (CFP), an enhanced cyan fluorescent protein (ECFP) or a red fluorescent protein (dsRED) or any other natural or genetically engineered fluorescent protein of those listed above. In yet further embodiments, the reconstituted fluorescent proteins may comprise of a mixture of fragments from the same or a combination any of the above listed fluorescent proteins.

In an embodiment where the fluorescent protein is EGFP, the EGFP protein is split into an alpha fragment (approximately amino acids 1-158) and a beta fragment (approximately amino acids 159-239). The alpha fragment contains a mature chromophore, which does not fluoresce alone, but is primed to fluoresce when paired with the beta fragment. Because the chromophore is preformed, it can immediately fluoresce. Importantly, the alpha and beta fragments do not reassociate or fluoresce in the absence facilitated association. In addition, the reassembled EGFP has an excitation/emission maxima that is red shifted to 490/524 nm, as compared to 488/507 nm for EGFP. Furthermore, the reassembled EGFP described herein is stabilized in the presence of Mg²⁺.

In an alternative embodiment of the invention, the activated split-polypeptide fragments can comprise fragments of an active enzyme, which can be detected using an enzyme activity assay. In such an embodiment, the enzyme activity is detected by a chromogenic or fluorogenic reaction. In one embodiment, the enzyme is dihydrofolate reductase or β-lactamase

Another aspect of the invention is an activated split-polypeptide molecule. In one embodiment, the molecule comprises at least two activated split-polypeptide fragments, each coupled to a nucleic acid binding moiety or nucleic acid binding motif. Nucleic acid binding moieties can be for example but are not limited to, nucleic acids such as DNA, RNA, and nucleic acid analogues such as, PNA, LNA and other analogues and oligonucleotides, which are specific for a desired nucleic acid target. In one embodiment, the nucleic acid binding moieties are oligonucleotides. In another embodiment, the nucleic acid binding moieties can be nucleic acid binding proteins, polypeptides or peptides. The nucleic acid binding moieties are coupled to at least two activated split-polypeptide fragments, and their association with, a target nucleic acid in close proximity facilitates the immediate formation of the active protein and immediate signal production. Where the activated split-polypeptide molecule comprises activated split-fluorescent fragments, the close association of the activated fluorescent fragments results in immediate fluorescence. The nucleic acid-binding moieties may associate with the target nucleic acid by functioning independently or cooperate to bind at a single site. In one embodiment, the target nucleic acids can be, for example, DNA, RNA, PNA or analogues or variants of nucleic acids.

In one embodiment of the present invention, nucleic acid binding moieties are conjugated to the activated split-polypeptide fragments via flexible linkers. In one embodiment a linker is biotin-streptavidin chemistry (see, for example, FIG. 1). In such an embodiment the two fluorescent fragments may be expressed with extra cysteine residues at the C- and N-termini, respectively, for biotinylation with the sulfhydryl-reactive reagent, biotin-HPDP. The C- and N-terminally biotinylated polypeptides can be then coupled with biotinylated nucleic acid binding moieties, for example oligonucleotides via streptavidin. Streptavidin, a high-affinity biotin-binding protein acts as a linker. In alternative embodiments, modification of the flexible linkers comprise changing the N-terminal amino acid and/or the C-terminal amino acid of each polypeptide to cysteine, and a thiol group at the 3′ or 5′ end of the nucleic acid binding moiety (or oligonucleotide) to allow coupling to the N-terminal and/or C-terminal cysteine.

In an alternative embodiment, the nucleic acid binding moieties coupled to the fluorescent protein fragments of the present invention may be other nucleic acid binding molecules, as non-limiting examples, PNAs, aptamers, RNA etc. In another embodiment, the nucleic acid binding moieties may be RNA- or DNA-binding proteins. The fluorescent proteins may be two inactive fragments which are attached to nucleic acid-binding motifs, where the nucleic acid binding motifs may function independently or cooperate to bind at a single site. Re-association of the fluorescent protein into a full-length protein will only occur in the presence of a target binding site, such as the interaction of an RNA-binding protein to its cognate binding site(s) on the RNA. This interaction will bring together the two halves of the fluorescent protein, allowing for signal detection.

Another aspect of the invention is an activated split-polypeptide molecule which comprises at least two activated split-polypeptide fragments, each coupled to a binding motif of a non-nucleic acid analyte. Such non-nucleic acid binding motifs can be for example but are not limited to, proteins, polypeptides or peptides. In other embodiments, the binding motif for a non-nucleic acid analyte can be, for example, a biomolecule, organic molecule or an inorganic molecule. In such an embodiment, the target analyte can be, for example, a biomolecule, inorganic molecule or organic molecule, or variants thereof.

When a fluorescent protein is used, it can be selected from a group comprising; green fluorescent protein (GFP), GFP-like fluorescent proteins, (GFP-like); enhanced green fluorescent protein (EGFP); yellow fluorescent protein (YFP); enhanced yellow fluorescent protein (EYFP); blue fluorescent protein (BFP); enhanced blue fluorescent protein (EBFP); cyan fluorescent protein (CFP); enhanced cyan fluorescent protein (ECFP); and red fluorescent protein (dsRED) and variants thereof.

In one embodiment, the activated split-polypeptide molecule provides methods for the real-time detection of nucleic acid molecules. Target nucleic acid molecules can be DNA, RNA as well as nucleic acid analogues. Target nucleic acids can be single or double stranded. In one some embodiments, the target nucleic acid can be amplified prior to exposure to the split-fluorescent molecule. For example, rolling circle amplification (RCA) can be used to generate a single-stranded DNA target with a multiplicity of the same hybridization sites, which bind to the probes of the complementation complex.

In one embodiment, the binding moieties bind to two adjacent sequences on the target nucleic acid, such that one nucleic acid binding moieties binds to a first target sequence and the second nucleic acid binding moiety binds to a second target sequence. In this embodiment, the adjacent sequences are close enough to each other to allow the first and second polypeptides to interact when both binding moieties are bound to the target, allowing complementation of the fluorescent fragments. This embodiment provides for detection of single-stranded and double-stranded target nucleic acids. For detection of double stranded targets, the single-stranded probes interact with the double-stranded target to form a triplex.

In an alternative embodiment, the both nucleic acid binding moieties are nucleic acids or oligonucleotides, and bind to the same sequence on a single-stranded target nucleic acid, forming a triplex. In this embodiment, complementation of the fluorescent fragment occurs when both binding moiteis interact with the same sequence on to the nucleic acid target.

In embodiments providing for formation of a triplex, the probe can be an oligonucleotide or a polypeptide. Preferred triplex-forming oligonucleotides are GC-rich. A preferred triplex is a purine triplex, consisting of pyrimidine-purine-purine.

In one embodiment, the present invention provides methods for real-time detection of the presence and/or quantity of target nucleic acid present in a sample. A sample containing a target nucleic acid is contacted under hybridization conditions with the split fluorescent molecule, with complementation of split fluorescent fragments and immediate production of fluorescence occurring when the nucleic acid binding moieties associate with the target nucleic acid. The presence and/or quantity of fluorescence is indicative of the presence and/or quantity of the target nucleic acid.

The present invention also provides methods for isolating a target nucleic acid in a sample, even in the presence of non-target sequences.

In another embodiment, the methods of the invention allows for real-time nucleic acid diagnostics. In particular, the detection of pathogen nucleic acid in a sample. In one embodiment, nucleic acid diagnostics as be used for the real-time detection of viral nucleic acids. In such an embodiment, the molecule of the present invention is designed so that the split fluorescent protein is bound to nucleic acid binding moieties or oligonucleotides that are specific for a particular viral nucleotide sequence or nucleotide sequence aberration due the viral nucleotide sequence.

In an alternative embodiment, the molecule of the present invention allows for the immediate detection of changes in nucleic acid hybridization. For example, in the presence of target nucleic acid, the two halves of the activated split-polypeptides associate to immediately form the active protein and therefore signal production in real-time. In particular, the immediate production of a fluorescent signal where the split-polypeptide fragments of the molecule comprise activated split-fluorescent fragments, However, if target nucleic acid becomes unavailable, such as in the presence of an excess of competitive inhibitor, the active protein disassembles and the signal dissipates and is no longer detected. The disassociation can be detected by a reduction in signal and/or fluorescence and such detection is immediate. The immediacy of detection upon disassociation is currently unavailable in the molecules in the art.

In another embodiment, the present invention provides methods for real-time immediate detection of hybridization of the oligonucleotides that serve as nucleotide binding moieties conjugated to activated split-polypeptide fragments. For example, localized heating (as described in Hamad-Schifferli et al., Nature, vol. 415, 10 Jan. 2002, herein incorporated by reference in its entirety) may be used to denature the bound oligonucleotides, thus shutting off fluorescence. The protein fragments of the present invention are unique in that upon disassociation the signal of the active protein is immediately quenched or ameliorated. They are also unique in that if the oligonucleotides are allowed to reassociate the signal is immediately re-established. The use of the present molecule in this embodiment allows for one to efficiently conduct and record results from various assays where multiple on-off cycling is required and allows for real time optical visualization of nucleic acid hybridization events. Further, the methods of the invention enable screening of agents which interrupt or promote hybridization and/or interfere with nucleic acid hybridization cycling events.

In another embodiment, the present invention allows for the real-time detection of gene mutations, polymorphisms, or aberrations in an individual or subject. A biological sample is isolated from an individual and DNA and/or RNA is extracted. The molecule of the present invention is designed so that the activated split-polypeptide fragments are bound to oligonucleotides that are specific for the particular mutation, polymorphism or aberration one is trying to detect. Alternatively, a pool of molecules may be used whereby many mutations, polymorphisms, or aberrations may be detected. In this embodiment, the oligonucleotides attached to the activated split-polypeptide fragments are complementary for each other and thus the baseline is the signal from the active protein. The DNA and/or RNA from the sample is then contacted to the molecule(s). If the individual from which the sample was obtained has the particular mutation or polymorphism, it will compete with the split-polypeptide molecule and reduce the active protein signal. The individual's DNA and/or RNA may be amplified prior to contact with the activated split-polypeptide molecule. This is particularly useful in the detection of single nucleotide polymorphisms of know polymorphisms. The present molecule allows for sensitive detections due to the immediacy of signal and/or fluorescent production.

In a similar embodiment, the present invention allows for the real-time detection of a analyte, in particular non-nucleic acid analyte, in a biological sample from an individual. A biological sample is isolated from a subject comprising the target analyte. In some embodiments, the target analyte can be extracted. The molecule of the present invention is designed so that the activated split-polypeptide fragments are conjugated to binding motifs specific to the analyte trying to detect. Alternatively, a sample comprising a pool of molecules or analytes may be used where one or more analytes may be detected. In this embodiment, the binding motif to the analyte is attached to the activated split-polypeptide fragments is specific to the analyte to be tested and is then contacted to the biological sample containing the analyte. If the subject from which the sample was obtained has the particular analyte, the split-polypeptide fragments will reassociate rendering the activated split-polypeptide molecule. This is particularly useful in the detection of single and multiple analytes in a sample, particularly when the detector proteins are fragments of fluorescent proteins, and when the fragments are from different fluorescent proteins whit different fluorescent spectra. The present molecule allows for sensitive detections due to the immediacy of signal and/or fluorescent production.

In another embodiment, the present invention provides kits suitable for detecting the presence and/or amount of a target nucleic acid or target non-nucleic acid analyte in a sample. In one embodiment, the kits comprise at least the components of the activated split-fluorescent protein molecule, namely the first fluorescent fragment comprising a preformed chromophore and a second fluorescent protein fragment which complements with the first fragment for immediate fluorescence. In alternative embodiments, the kit comprises at least the components of an activated split-polypeptide molecule where the activated split-polypeptide reconstitutes to from an enzyme with chromogenic activity. In some embodiments, nucleic acid binding moieties or binding motif of the analyte are already associated with the activated split-polypeptide protein fragments. In alternative embodiments, the split-polypeptides fragments may be biotinylated with the sulfhydryl-reactive reagent, biotin-HPDP. In such kits, the kit comprises the reagents for coupling of the users own binding moiety of interest with the split-polypeptide fragments. In some embodiments, the kits also comprise reagents suitable for capturing and/or detecting the present or amount of target nucleic acid or target non-nucleic acid analyte in a sample. The reagents for detecting the present and/or amount of target nucleic acid can include enzymatic activity reagents or an antibody specific for the assembled protein. The antibody can be labeled.

### BRIEF DESCRIPTION OF THE DRAWINGS

FIG. 1: Design of the fluorescent protein-based optical nano-switch regulated by DNA hybridization. Fluorescent protein (EGFP) is dissected into two non-fluorescent fragments, one of which contains pre-formed chromophore capable of bright fluorescence within a full-size protein. Both protein fragments are linked to complementary oligonucleotides via biotin-streptavidin interactions; while streptavidin can bind up to four molecules of biotin, in our protocol we ensure 1:1:1 ratio of protein/streptavidin/oligonucleotide complex. In a mixture, these two nucleoprotein constructs merge by sequence-specific duplex DNA formation, which triggers complementation of the large and small EGFP fragments resulting in fast development of fluorescence (switch-on state). Addition of the excess of one of the oligonucleotides displaces corresponding nucleoprotein component and shuts down fluorescence (switch-off state).

FIGS. 2A-2D: Structure of the large EGFP fragment (1-158 N-terminal amino acids) analyzed by DMD simulations. FIG. 2A. Potential energy: of the large EGFP fragment as a function of temperature (standard deviations are shown by the error bars). The protein folding occurs in the narrow temperature range close to the transition point T_{F}˜0.60. FIG. 2B. A trajectory of potential energy as a function of simulation time at T=0.59 demonstrating that near T_{F} the protein structure rapidly changes between folded (lower lines) and unfolded (upper lines) states. FIG. 2C. Backbone representation of ten folded and aligned structures of the large EGFP fragment that were obtained in DMD simulations. The segment from 62 to 70 amino acids, containing the chromophore-forming amino acids (T66, Y67 and G68), is colored blue. The C-terminus of this polypeptide is very flexible due to a small number of contacts with the rest of the molecule so that the alignment was made by omitting the corresponding amino acids. FIG. 2D. Backbone alignment of one of the DMD-folded large EGFP fragments (blue) and the full-length EGFP (yellow). Here, the chromophore-forming residues of both polypeptides are shown in red. FIG. 2E. The root-mean-square deviation (RMSD) of each residue in the folded large EGFP fragment. The chromophore-forming residues are in shaded region and their spatial arrangement is essentially fixed, with deviation being less then 2.

FIGS. 3A-3C: Spectral features of cloned EGFP fragments. FIG. 3A. SDS-PAGE analysis (15% PAGE) of the exemplary protein samples containing the large (lanes 1) and small (lanes 2) EGFP fragments overexpressed in E. Coli and isolated using the intein self-splicing technology. Lane M corresponds to the protein molecular weight ladder. Large and small EGFP fragments are seen as ˜15 kD and ˜10 kD bands, respectively (marked with red asterisks). While the small EGFP fragment is practically pure, the large EGFP fragment is somewhat contaminated by intein (˜25 kD) and unsplit fusion (˜40 kD). FIG. 3B. Absorption spectra of protein samples with the large (curves 1) and small (curves 2) EGFP fragments. The protein samples are represented by two typical spectra (correspond to 40 μM of EGFP fragments in PBS buffer, pH 7.4) showing the range of their absorption at each wavelength. Curve 3: 40 μM streptavidin; inset: 2 μM EGFP. FIG. 3c. Fluorescence excitation (curve 1) and emission (curve 2) spectra of the large EGFP fragment (2 μM in PBS buffer, pH 7.4).

FIGS. 4A-4B: Assembly and performance of the DNA hybridization-driven optical nano-switch. FIG. 4A. Gel-shift assay (SDS-10% PAGE) of binding the increased amounts of biotinylated EGFP fragments with fixed amount of streptavidin (2 μg; 60-kD band). Red arrows indicate the protein amounts resulting in 1:1 complexes (70-75-kD bands), which correspond to ≧70% yield of biotinylation. FIG. 4B. Gel-shift assay (10% PAGE) demonstrating the formation of 1:1:1 tripartite molecular constructions depicted in FIG. 1 and comprising the large or small EGFP fragment, streptavidin and a corresponding oligonucleotide. Lanes 1 and 2: biotinylated oligo 1 in the absence (1) or presence (2) of the large EGFP fragment coupled to streptavidin; lanes 3 and 4: biotinylated oligonucleotide 2 in the absence (3) or presence (4) of the small EGFP fragment coupled to streptavidin; M: 20-bp size marker. Red arrow marks the position of the required oligonucleotide-protein complexes that are strongly shifted upward, as expected. FIG. 4C. Fluorescence spectra of intact EGFP (1) and of the split EGFP-based protein complex re-assembled by DNA hybridization from the tripartite molecular constructions (2), each taken at ˜200 nM concentrations in PBS buffer, pH 7.4 (spectra recorded 20 min after mixing). Curve 3: same as sample 2 plus 100-fold excess of one of the two complementary oligonucleotides (non-biotinylated oligo 1). Curve 4: control containing both EGFP fragments coupled to streptavidin but without oligonucleotides. Inset shows the time course of the fluorescence development in sample 2 recorded at 524 nm. FIG. 4D. Effect of Mg⁺² cations on intact EGFP (blue) and on the re-assembled split EGFP complex containing duplex DNA (purple). Column 1: no Mg⁺²; columns 2 and 3:2 min and 3 hr after addition of 2 mM Mg⁺².

FIG. 5: Full-length EGFP is more stable than its large fragment. The graph shows folding thermodynamics of the large EGFP fragment (aka alpha domain) as compared to full-length EGFP; it is clear that the latter has a higher transition temperature T_{F}. Evidently, the increase in stability is a result of interactions between the large and small EGFP fragments. Thus, the presence of the smaller EGFP domain substantially stabilizes the fold of the full-size protein. Folded EGFP structure: X-ray structure (PDB code 1c4f; S65T GFP mutant/pH 4.6); we consider this conformation as a native EGFP fold because differences between this structure and some other EGFP X-ray structures are small. For instance, the rmsd between PDB 1c4f and PDB 1emg (S65T GFP mutant/pH 8.0) is only 0.18 Å.

FIG. 6: Full-length EGFP has two folding-unfolding intermediate states. These two graphs show the quasi-equilibrium unfolding of EGFP studied by quasi-equilibrium heating DMD simulations using the Berendsen's thermostat³¹. Starting from folded state, the temperature of the protein system is slowly increased from Tₗ=0.6<T_{F} to Tₕ=0.8>T_{F}. We performed 10 unfolding simulations of EGFP. A typical trajectory is shown (top); two unfolding intermediate states, I₁^{U} and I₂^{U}, are observed along the averaged folding pathway (bottom). Similar results were obtained for 10 quasi-equilibrium EGFP folding simulations.

FIG. 7: Unfolding intermediate I₁^{U} (left snapshot) corresponds to the unfurling of the N-terminal β-strands, and the second unfolding intermediate I₂^{U} (right snapshot) corresponds to the unfolding of almost the entire larger EGFP domain (light color) with its C-terminal interacting with the smaller EGFP domain (dark color)

### MODES FOR CARRYING OUT THE INVENTION

The inventors have discovered a novel method for rapid real-time protein complementation involving the production of activated split-polypeptide fragments in vitro. The methods also relate to real-time detection of nucleic acid molecules and nucleic acid hybridization, or non-nucleic acid analytes using protein complementation of activated split-polypeptide fragments (which can also be referred to as a biomolecular constructs). In the present invention, the inventors have discovered methods to produce activated split-polypeptide fragments in a ready state, wherein if in close proximity with similarly activated complementary split-polypeptide fragment(s), an active protein is immediately formed. Also disclosed are novel methods to split fluorescent proteins into activated split-fluorescent proteins. The production of activated split-polypeptide fragments in a ready state and in the active configuration enables real-time protein complementation, whereas previous protein complementation methods used inactive split-polypeptide fragments that required reconfiguration in order to form the active protein. The methods of the present invention using activated split polypeptide fragments enable real-time protein complementation that is rapid, sensitive and reversible.

In one embodiment, the methods of the present invention comprises expressing a nucleic acid encoding a first and second polypeptide fragment in a microbial host cell to form inclusion bodies. The inclusion bodies enable proper protein folding and thus contain proteins which are folded in a state that more closely mirrors an in vivo state than traditional methods of purification. Other means can be used based upon known techniques such as cells with vesicles. For example, inclusion bodies enable the production of split-polypeptide proteins in an activated ready-state. The inclusion bodies are harvested, lysed and resolubilized to obtain the split-polypeptide protein fragments.

### Activated Split-Polypeptide Fragments.

The activated spit-polypeptide fragments can be any polypeptides which associate when brought in to close proximity to generate a protein, which can be detected by any means which allows recognition of the assembled polypeptide fragments but not the individual polypeptides fragments. In one embodiment of the current invention, the methods encompass the design of split-polypeptide fragments so that they are active immediately upon their reconstitution.

The activated split-polypeptide fragments can be any polypeptide which associate when brought in to close proximity to generate an active protein, which can be detected by any means which allows recognition of the assembled active protein but not the individual polypeptides. For example, the two polypeptides may re-associate to generate a protein with enzymatic activity, to generate a protein with chromogenic or fluorogenic activity, or which create a protein recognized by an antibody. Furthermore, they are designed so that they are in the active state and primed (i.e. in a ready-state) for reconstitution of the active protein in order to minimize any lag time that is traditionally seen with protein complementation in vitro and in vivo.

In one embodiment the activated split-polypeptide fragments are fluorescent proteins or polypeptides. In such an embodiment, one of the activated split fluorescent protein fragments contains a mature preformed chromophore that is primed and in the ready-state for immediate fluorescence upon complementation with its cognate activated split-fluorescent fragment(s). For example, using inclusion bodies containing such a split fluorescent fragment comprises about half of a fully folded fluorescent protein with a correctly folded a mature chromophore that does not fluoresce alone, but is primed to fluoresce upon association with its cognate pair.

In one such embodiment, the assembled protein is green fluorescent protein (GFP), a modified GFP such as EGFP or GFP-like fluorescent proteins or any other natural or genetically engineered fluorescent protein known by persons skilled in the art, including but not limited to CFP, YFP, and RFP.

In some embodiments, the cognate non-fluorescent polypeptide fragment which combines with the mature chromophore-containing split-fluorescent fragment can comprise of more than one active non-fluorescent fragment. Such activated non-fluorescent polypeptides are usually produced by splitting the coding nucleotide sequence of one fluorescent protein at an appropriate site and expressing each nucleotide sequence fragment independently. The activated split-fluorescent protein fragments may be expressed alone or in fusion with one or more protein fusion partners.

In one embodiment of the invention, the reconstituted active protein comprises of activated split-EGFP fragments, wherein the first fragment is an N-terminal fragment of EGFP comprising a continuous stretch of amino acids from amino acid number 1 to approximately amino acid number 158. A C-terminal cysteine may be added to this fragment to aid in the conjugation of various nucleic acid binding motifs post expression. The second activated split-EGFP fragment is a continuous stretch of amino acids from approximately amino acid number 159 to amino acid number 239. A N-terminal cysteine may also be added.

Amino acid 1 is meant to indicate the first amino acid of EGFP. Amino acid 239 is meant to indicate the last amino acid of the GFP. All residues are numbered according to the numbering of wild type A. victoria GFP (GenBank accession no. M62653; SEQ ID NO 7) and the numbering also applies to equivalent positions in homologous sequences. Thus, when working with truncated GFPs (compared to wild type GFP) or when working with GFPs with additional amino acids, the numbering must be altered accordingly.

Green Fluorescent Protein (GFP) is a 238 amino acid long protein derived from the jellyfish Aequorea Victoria (see mRNA sequence at SEQ ID NO: 8). However, fluorescent proteins have also been isolated from other members of the Coelenterata, such as the red fluorescent protein from Discosoma sp. (Matz, M. V. et al. 1999, Nature Biotechnology 17: 969-973), GFP from Renilla reniformis, GFP from Renilla Muelleri or fluorescent proteins from other animals, fungi or plants (U.S. Pat. No. 7,109,315). GFP exists in various modified forms including the blue fluorescent variant of GFP (BFP) disclosed by Heim et al. (Heim, R. et al, 1994, Proc. Natl. Acad. Sci. 91:26, pp 12501-12504) which is a Y66H variant of wild type GFP; the yellow fluorescent variant of GFP (YFP) with the S65G, S72A, and T203Y mutations (WO98/06737); the cyan fluorescent variant of GFP (CFP) with the Y66W color mutation and optionally the F64L, S65T, N1461, M153T, V163A folding/solubility mutations (Heim, R., Tsien, R. Y. (1996) Curr. Biol. 6, 178-182). The most widely used variant of GFP is EGFP with the F64L and S65T mutations (WO 97/11094 and WO96/23810) and insertion of one valine residue after the first Met. The F64L mutation is the amino acid in position 1 upstream from the chromophore. GFP containing this folding mutation provides an increase in fluorescence intensity when the GFP is expressed in cells at a temperature above about 30° C. (WO 97/11094). All of the above mentioned fluorescent proteins and functional fragments thereof are encompassed for use in the present invention. Also encompassed are those fluorescent proteins known to those of skill in the art, and fragments thereof.

In alternative embodiments, the reconstituted fluorescent protein may comprise of activated split-fluorescent fragments selected from a group comprising; green fluorescent protein (GFP), enhanced green fluorescent protein (EGFP), green-fluorescent-like proteins; yellow fluorescent protein (YFP), enhanced yellow fluorescent protein (EYFP), blue fluorescent protein (BFP), enhanced blue fluorescent protein (EBFP), cyan fluorescent protein (CFP), enhanced cyan fluorescent protein (ECFP) or a red fluorescent protein (dsRED), where one of the fragments in the reconstituted fluorescent protein contains a mature preformed chromophores. All of the above mentioned fluorescent proteins and fragments thereof that will result in a fluorescing fluorescent protein are encompassed for use in the present invention. Also encompassed are those fluorescent proteins known to those of skill in the art, and fragments and genetically engineered proteins thereof.

In alternative embodiments, the reassembled fluorescent protein may comprise activated split fluorescence fragments from different and spectrally distinct fluorescent proteins. The reconstituted active fluorescent protein may have a distinct and/or unique spectral characteristics depending on the activated split-fluorescent fragments used for complementation. For example, multicolor fluorescence complementation has been achieved by reconstituting fragments from different fluorescent proteins for multicolor biomolecular fluorescence complementation (multicolor BiFC) (see Hu et al, Nature Biotechnology, 2003; 21; 539-545; Kerppola, 2006, 7; 449-456, Hu, et al, Protein-Protein Interactions (Ed. P. Adams and E. Golemis), Cold Spring Harbor Laboratory Press. 2005, herein incorporated by reference in its entirety) Encompassed for use in the present invention are the use of activated split-fluorescent fragments from multiple fluorescent proteins for multicolor real-time fluorescence, wherein one of the fragments contains a pre-formed mature chromophore.

In one embodiment, the fluorescent protein is detectable by flow cytometry, fluorescence plate reader, fluorometer, microscopy, fluorescence resonance energy transfer (FRET), by the naked eye or by other methods known to persons skilled in the art. In an alternative embodiment, fluorescence is detected by flow cytometry using a florescence activated cell sorter (FACS) or time lapse microscopy.

In another embodiment of the invention, the activated split-polypeptide fragments associated in close proximity to form an assembled, active enzyme, which can be detected using an enzyme activity assay. Preferably, the enzyme activity is detected by a chromogenic or fluorogenic reaction. In one preferred embodiment, the enzyme is dihydrofolate reductase (DHFR) or β-lactamase.

In another embodiment, the enzyme is dihydrofolate reductase (DHFR). For example, Michnick et al. have developed a “protein complementation assay” consisting of N- and C-terminal fragments of DHFR, which lack any enzymatic activity alone, but form a functional enzyme when brought into close proximity. See e.g. U.S. Pat. Nos. 6,428,951, 6,294,330, and 6,270,964, which are hereby incorporated by reference. Methods to detect DHFR activity, including chromogenic and fluorogenic methods, are well known in the art.

In alternative embodiments, other split polypeptides can be used. For example, enzymes that catalyze the conversion of a substrate to a detectable product. Several such systems for split-polypeptide reassemblies include, but are not limited to reassembly of; β-galactosidase (Rossi et al, 1997, PNAS, 94; 8405-8410); dihydrofolate reductase (DHFR) (Pelletier et al, PNAS, 1998; 95; 12141-12146); TEM-1 β-lactamase (LAC) (Galarneau at al, Nat. Biotech. 2002; 20; 619-622) and firefly luciferase (Ray et al, PNAS, 2002, 99; 3105-3110 and Paulmurugan et al, 2002; PNAS, 99; 15608-15613). For example, split β-lactamase has been used for the detection of double stranded DNA (see Ooi et al, Biochemistry, 2006; 45; 3620-3525). Encompassed for use in the present invention are the use of activated split polypeptide fragments for real-time signal detection, wherein the fragments are in a fully folded mature conformation enabling rapid signal detection upon complementation.

In another embodiment of the invention, association of activated split-polypeptide fragments can form an assembled protein which contains a discontinuous epitope, which may be detected by use of an antibody which specifically recognizes the discontinuous epitope on the assembled protein but not the partial epitope present on either individual polypeptide. One such example of a discontinuous epitope is found in gp120 of HIV. These and other such derivatives can readily be made by the person of ordinary skill in the art based upon well known techniques, and screened for antibodies that recognize the assembled protein by neither protein fragment on its own.

In another embodiment of the invention, the activated split-polypeptides can be molecules which interact to form an assembled protein. For example, the molecules may be protein fragments, or subunits of a dimer or multimer.

The nucleic acid sequence and codons encoding the split-polypeptide fragments of interest may be optimized, for example, converting the codons to ones which are preferentially used in a desired system. For example in mammalian cells. Optimal codons for expression of proteins in non-mammalian cells are also known in the art, and can be used when the host cell is a non-mammalian cell (for example in insect cells).

The activated split-polypeptides of the present invention can comprise any additional modifications which are desirable. For example, in one embodiment, the activated split-polypeptides can also comprise a flexible linker, which is coupled to a nucleic acid binding moiety.

### Expression of Fluorescent Fragments and Inclusion Bodies

There exist a large number of publications which describe the recombinant production of proteins in microorganisms/prokaryotes via the inclusion bodies route. Examples of such reviews are Misawa, S., et al., Biopolymers 51 (1999) 297-307; Lilie, H., Curr. Opin. Biotechnol. 9 (1998) 497-501; Hockney, R. C., Trends Biotechnol. 12 (1994) 456-463.

The peptides according to the invention are overexpressed in microorganisms and/or prokaryotes. Overexpression leads to the formation of inclusion bodies. Methionine encoded by the start codon is mainly removed during the expression/translation in the host cell. General methods for overexpression of proteins in microorganisms/prokaryotes have been well-known in the state of the art. Examples of publications in the field are Skelly, J. V., et al., Methods Mol. Biol. 56 (1996) 23-53; Das, A., Methods Enzymol. 182 (1990) 93-112; and Kopetzki, E., et al., Clin. Chem. 40 (1994) 688-704.

As used herein, overexpression in prokaryotes means expression using optimized expression cassettes (U.S. Pat. No. 6,291,245) with promoters such as the tac or lac promoter (EP-B 0 067 540). Usually, this can be performed by the use of vectors containing chemical inducible promoters or promoters inducible via shift of temperature. One of the useful promoters for E. coli is the temperature-sensitive lambda-PL promoter (EP-B 0 041 767). A further efficient promoter is the tac promoter (U.S. Pat. No. 4,551,433). Such strong regulation signals for prokaryotes such as E. coli usually originate from bacteria-challenging bacteriophages (see Lanzer, M., et al., Proc. Natl. Acad. Sci. USA 85 (1988) 8973-8977; Knaus, R., and Bujard, H., EMBO Journal 7 (1988) 2919-2923; for the lambda T7 promoter: Studier, F. W., et al., Methods Enzymol. 185 (1990) 60-89); for the T5 promoter: EP-A 0 186 069; Stuber, D., et al., System for high-level production in Escherichia coli and rapid application to epitope mapping, preparation of antibodies, and structure-function analysis; In: Immunological Methods IV (1990) 121-152).

By the use of such overproducing prokaryotic cell expression systems the peptides according to the invention are produced at levels at least comprising 10% of the total expressed protein of the cell, and typically 30-40%, and occasionally as high as 50%.

“Inclusion bodies” (IBs), as used herein, refer to an insoluble form of polypeptide's recombinantly produced after overexpression of the encoding nucleic acid in microorganisms/prokaryotes.

Solubilization of the inclusion bodies is preferably performed by the use of aqueous solutions with pH values of about 9 or higher. Most preferred is a pH value of 10.0 or higher. It is not necessary to add detergents or denaturing agents for solubilization. The optimized pH value can be easily determined. It is obvious that there exists an optimized pH range as strong alkaline conditions might denature the polypeptides. This optimized range is found between pH 9 and pH 12.

Nucleic acids (DNA) encoding the fluorescent peptides can be produced according to the methods known in the state of the art. It is further preferred to extend the nucleic acid sequence with additional regulation and transcription elements, in order to optimize the expression in the host cell. A nucleic acid (DNA) that is suitable for the expression can preferably be produced by chemical synthesis. Such processes are familiar to persons skilled in the art and are described for example in Beattie, K. L., and Fowler, R. F., Nature 352 (1991) 548-549; EP-B 0 424 990; Itakura, K., et al., Science 198 (1977) 1056-1063. It may also be expedient to modify the nucleic acid sequence of the peptides according to the invention.

Such modifications are, for example but not limited to; modification of the nucleic acid sequence in order to introduce various recognition sequences of restriction enzymes to facilitate the steps of ligation, cloning and mutagenesis; modification of the nucleic acid sequence to incorporate preferred codons for the host cell; extension of the nucleic acid sequence with additional regulation and transcription elements in order to optimize gene expression in the host cell.

The codons used to synthesize the protein of interest may be optimized, converting them to codons that are preferentially used in a desired system. For example in mammalian cells. Optimal codons for expression of proteins in non-mammalian cells are also known, and can be used when the host cell is a non-mammalian cell (for example in insect cells).

### Split-Polypeptide Molecule.

Also encompassed in the present invention is an activated split-polypeptide molecule, also referred to as biomolecular conjugate, produced by the methods described herein. In one embodiment, the activated split-polypeptide molecule comprises a split-polypeptides of an enzyme with chromogenic or fluorogenic activity. In one embodiment, the enzyme is dihydrofolate reductase or β-lactamase or luciferase. In one embodiment, the fluorescent protein is GFP or GFP-like fluorescent proteins.

In some embodiments, the activated split-polypeptide of the molecule further comprises a nucleic acid binding motif or nucleic acid binding moieties. In the presence of a target nucleic acid, the binding of a nucleic acid binding moieties to the nucleic acid target sequence facilitates the association of the activated split-polypeptide fragment to form an active protein.

In alternative embodiments, the activated split-polypeptide of the molecule further comprises a binding motif for a non-nucleic acid analyte. In the presence of a target analyte, typically a non-nucleic acid analyte, the binding of the analyte binding motif to the target analyte facilitates the association of the activated split-polypeptide fragment to from an active protein.

In another embodiment, the activated split-polypeptide molecule is a split-fluorescent molecule. In such an embodiment, the molecule comprises at least two activated split fluorescent fragments selected from the group consisting of GFP, GFP-like fluorescent proteins, fluorescent proteins, and variants thereof. One of the split-fluorescent fragments comprises a mature preformed chromophore which is active by in a non-fluorescent state in the dissociated fragment. The activated fluorescent fragments, when associated with each other contain the full complement of beta-strands necessary for fluorescence, but are not fluorescent by themselves. Each of the activated split-fluorescent fragments of the molecule further comprise nucleic acid binding motif. The binding of the nucleic acid binding motifs to a target nucleic acid facilitates the association of at least two active split-fluorescent fragments and reconstitution of the active fluorescent protein and fluorescent phenotype in real time.

### Nucleic Acid Binding Moieties.

The nucleic acid binding moiety of each split-polypeptide molecule can be any molecule which allows binding to a target nucleic acid. In some embodiments, the nucleic acid binding moiety includes nucleic acids, nucleic acid analogues, and polypeptides. In one embodiment, the nucleic acid binding moiety is an oligonucleotide. The nucleic acid binding moiety of a given pair of activated split-polypeptide fragment can be of the same kind of molecule, for example oligonucleotides, or they can be different, for example one split-polypeptide of a pair comprise an active protein can have an oligonucleotide nucleic acid binding moiety, and the other member of the pair can have a polypeptide nucleic acid binding moiety.

The nucleic acid binding moiety can be any molecule that can be coupled to another molecule, such as a polypeptide, and are capable of binding to a target nucleic acid in close proximity. In one embodiment, the nucleic acid binding moiety is a nucleic acid or nucleic acid analogue, such as an oligonucleotide. In another embodiment of the present invention, nucleic acid binding moieties are nucleic-acid binding polypeptide or proteins, which interacts with the target nucleic acid with high affinity. Nucleic acid analogues include, for example but not limited to, peptide nucleic acids (PNAs) pseudo-complementary PNA (pcPNA), locked nucleic acids, morpholin DNAs, phosphorothioate DNAs, and 2′-O-methoxymethyl-RNAs, locked nucleic acid (LNA) which is a nucleic acid analog that contains a 2′-O, 4′-C methylene bridge.

Nucleic acid binding moiety can bind to the same hybridization site on a single-stranded target, creating a triplex at the hybridization site. Alternatively, nucleic acid binding moieties can bind to closely adjacent hybridization sites on a single-stranded or double-stranded target nucleic acid, creating either a duplex or a triplex at each hybridization site, respectively.

In the embodiment where the nucleic acid binding moiety is a nucleic acid, the length of the nucleic acid binding moiety should be long enough to allow complementary binding to the nucleic acid target, and should allow one of the split-polypeptide fragments to interact with its corresponding split-polypeptide fragment(s) when both probe portions are bound to the same target nucleic acid. For example, the nucleic acid binding moiety probe can be 5-30 bases long. More preferably, 5-15 bases long.

In embodiments providing for formation of a triplex, the nucleic acid binding moiety can be any nucleic acid which allows triplex formation. Preferred triplex-forming oligonucleotides are GC-rich. A preferred triplex is a purine triplex, consisting of pyrimidine-purine-purine.

One preferred triplex-forming oligonucleotide is GC-rich. A preferred triplex is a purine triplex, consisting of pyrimidine-purine-purine.

Nucleic acid binding moiety can be selected from a group comprising; oligonucleotides; single stranded RNA molecules; and peptide nucleic acids (PNAs) including pseudocomplementary PNAs (pcPNA), locked nucleic acids (LNA) and other nucleic acid analogues.

In one embodiment, the nucleic acid binding moieties are oligonucleotides. Methods for designing and synthesizing oligonucleotides are well known in the art. Oligonucleotides are sometimes referred to as oligonucleotide primers.

Oligonucleotides useful in the present invention can be synthesized using established oligonucleotide synthesis methods. Methods of synthesizing oligonucleotides are well known in the art. Such methods can range from standard enzymatic digestion followed by nucleotide fragment isolation (see for example, Sambrook, et al., Molecular Cloning: A Laboratory Manual, Second Edition, Cold Spring Harbor, N.Y., (1989), Wu et al, Methods in Gene Biotechnology (CRC Press, New York, N.Y., 1997), and Recombinant Gene Expression Protocols, in Methods in Molecular Biology, Vol. 62, (Tuan, ed., Humana Press, Totowa, N.J., 1997), the disclosures of which are hereby incorporated by reference), to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method).

Many of the oligonucleotides described herein are designed to be complementary to certain portions of other oligonucleotides or nucleic acids such that stable hybrids can be formed between them. The stability of these hybrids can be calculated using known methods such as those described in Lesnick and Freier, Biochemistry 34:10807-10815 (1995), McGraw et al., Biotechniques 8:674-678 (1990), and Rychlik et al., Nucleic Acids Res. 18:6409-6412 (1990).

In one embodiment, the nucleic acid binding moieties are single stranded RNA molecules. Methods for designing and synthesizing single stranded RNA molecules are well known in the art.

In some embodiments, the nucleic acid binding moieties are peptide nucleic acids (PNAs), including pseudocomplementary PNAs (pcPNA). Methods for designing and synthesizing PNAs and pcPNAs are well known in the art. Peptide nucleic acids (PNAs) are analogs of DNA in which the backbone is a pseudopeptide rather than a sugar. Thus, their behavior mimics that of DNA and binds complementary nucleic acid strands. In peptide nucleic acids, the deoxyribose phosphate backbone of oligonucleotides has been replaced with a backbone more akin to a peptide than a sugar phosphodiester. Each subunit has a naturally occurring or non naturally occurring base attached to this backbone. One such backbone is constructed of repeating units of N-(2-aminoethyl)glycine linked through amide bonds.

PNA binds both DNA and RNA. The resulting PNA/DNA or PNA/RNA duplexes are bound with greater affinity and increased specificity than corresponding DNA/DNA or DNA/RNA duplexes. In addition, their polyamide backbone (having appropriate nucleobases or other side chain groups attached thereto) is not recognized by either nucleases or proteases, and thus PNAs are resistant to degradation by enzymes, unlike DNA and peptides. The binding of a PNA strand to a DNA or RNA strand can occur in either a parallel of anti-parallel orientation. PNAs bind to both single stranded DNA and double stranded DNA.

To address the sequence limitations of traditional PNAs, pseudocomplementary PNAs (pcPNAs) have been developed. In addition to guanine and cytosine, pcPNA's carry 2,6-diaminopurine (D) and 2-thiouracil instead of adenine and thymine, respectively pcPNAs exhibit a distinct binding mode, double-duplex invasion, which is based on the Watson-Crick recognition principle supplemented by the notion of pseudocomplentarity pcPNAs recognize and bind with their natural A, T, (U), or G, C counterparts. pcPNAs can be made according to any method known in the art. For example, methods for the chemical assembly of PNAs are well known (See: U.S. Pat. Nos. 5,539,082, 5,527,675, 5,623,049, 5,714,331, 5,736,336, 5,773,571 or 5,786,571, herein incorporated by reference).

Other embodiments of the invention provide nucleic acid binding moieties which are polypeptides or peptides. The polypeptide can be any polypeptide with a high affinity for the target nucleic acid. In this embodiment, the target nucleic acid can be a double-stranded, triple-stranded, or single-stranded DNA or RNA. In some embodiments, the polypeptides is a peptide, less than 100 amino acids, or a full length protein. The polypeptide's affinity for the target nucleic acid can in the low nanomolar to high picomolar range. Polypeptides can include polypeptides which contain zinc fingers, either natural or designed by rational or screening approaches. Examples of zinc fingers include Zif 2g8, Sp1, finger 5 of Gfi-1, finger 3 of YY1, finger 4 and 6 of CF2II, and finger 2 of TTK (PNAS (2000) 97: 1495-1500; J Biol Chem (20010 276 (21): 29466-78; Nucl Acids Res (2001) 29 (24):4920-9; Nucl Acid Res (2001) 29(11): 2427-36). Other polypeptides include polypeptides, obtained by in vitro selection, that bind to specific nucleic acids sequences. Examples of such aptamers include platelet-derived growth factor (PDGF) (Nat Biotech (2002) 20:473-77) and thrombin (Nature (1992) 355: 564-6. Yet other polypeptides are polypeptides which bind to DNA triplexes in vitro; examples include members of the heteronuclear ribonucleic particles (hnRNP) proteins such as hnRNP K, L, E1, A2/B1 and I (Nucl Acids Res (2001)29(11): 2427-36).

For split-polypeptide fragments which have a polypeptides as the nucleic acid binding moiety, the entire split-polypeptide fragment and nucleic acid binding moiety molecule can be encoded by a single construct, including the polypeptide portion, a linker and the nucleic acid binding moiety polypeptide. This construct can either be expressed in the cell or microinjected into the cell. These constructs can also be used for in vitro detection of a nucleic acid of interest.

### Nucleic Acid Targets

The method of the present invention can be used to detect the presence of a single-stranded nucleic acid target or a double-stranded nucleic acid, by generating a detectable signal associated with formation of the complementation complex.

The nucleic acid target can be any nucleic acid which contains hybridization sites for binding of the nucleic acid binding moiety associated to the activated split-polypeptide fragment. For example, the target nucleic acid can be DNA, RNA, or a nucleic acid analogue. The target nucleic acid can be single-stranded or double-stranded. The target nucleic acid can be detected in vivo or in vitro. In one embodiment, the method of the present invention is used to detect a target nucleic acid in vitro, and the activated split-polypeptides interact to generate an active protein with chromogenic and/or fluorogenic activity. In some embodiments, the polypeptides encode GFP, a modified GFP such as EGFP of GFP-like fluorescent proteins, or any other natural or genetically engineered fluorescent proteins including CFP, YFP, and RFP.

In another embodiment, the nucleic acid binding moieties bind to two adjacent sequences on the target nucleic acid, such that one nucleic acid binding moiety binds to one target sequence and the second nucleic acid binding moiety binds to another target sequence. In this embodiment, the adjacent sequences are close enough to each other to allow the associated activated split-polypeptide fragments to interact when their associated nucleic acid binding moieties are bound to the target, allowing assembly of the active protein. This embodiment provides for detection of single-stranded and double-stranded target nucleic acids. For detection of double stranded targets, the single-stranded probes interact with the double-stranded target to form a triplex.

Any nucleic acid target from a sample may be used in practicing the present invention, including without limitation eukaryotic, prokaryotic and viral DNA or RNA. In one embodiment, the target nucleic acid represents a sample of genomic DNA isolated from a patient. This DNA may be obtained from any cell source or body fluid. Non-limiting examples of cell sources available in clinical practice include blood cells, buccal cells, cervicovaginal cells, epithelial cells from urine, fetal cells, or any cells present in tissue obtained by biopsy. Body fluids include blood, urine, cerebrospinal fluid, semen and tissue exudates at the site of infection or inflammation. In another embodiment, the DNA is detected directly in the sample, without any additional purification. In another embodiment, the DNA is extracted from the cell source or body fluid using any of the numerous methods that are standard in the art. It will be understood that the particular method used to extract DNA will depend on the nature of the source. In certain embodiments, the amount of DNA to be extracted for use in the present invention is at least 5 pg (corresponding to about 1 cell equivalent of a genome size of 4×109 base pairs).

In one embodiment, the target nucleic acid can be amplified prior to exposure to the components of the complementation complex. Any method of amplifying a nucleic acid target can be used, including methods which generate a single stranded nucleic acid with a multiplicity of the same hybridization sites. The amplification reaction can be polymerase chain reaction (PCR), ligase chain reaction (LCR), strand displacement amplification (SDA), transcription mediated amplification (TMA), Qβ-replicase amplification (Q-beta), or rolling circle amplification (RCA).

In some embodiment, PCR is used to amplify the nucleic acid target.

Any polymerase which can synthesize the desired nucleic acid may be used. Preferred polymerases include but are not limited to Sequenase, Vent, and Taq polymerase. Preferably, one uses a high fidelity polymerase (such as Clontech HF-2) to minimize polymerase-introduced mutations.

In another embodiment, rolling circle amplification (RCA) is used to generate a single-stranded DNA target with a multiplicity of the same hybridization sites. Rolling circle amplification (RCA) is an isothermal process for generating multiple copies of a sequence. In rolling circle DNA replication in vivo, a DNA polymerase extends a primer on a circular template (Komberg, A. and Baker, T. A. DNA Replication, W. H. Freeman, New York, 1991). The product consists of tandemly linked copies of the complementary sequence of the template. RCA is a method that has been adapted for use in vitro for DNA amplification (Fire, A. and Si-Qun Xu, Proc. Natl. Acad. Sci. USA, 1995, 92:4641-4645; Lui, D., et al., J. Am. Chem. Soc., 1996, 118:1587-1594; Lizardi, P. M., et al., Nature Genetics, 1998, 19:225-232; U.S. Pat. No. 5,714,320 to Kool).

In another embodiment, the split-polypeptide molecule comprising a nucleic acid binding motif can be used for the detection of nucleic acid in immunoRCA (immuno-rolling circle amplification) and immunoPCR. In such an embodiment, the nucleic acid binding motifs components of the split-polypeptide molecule facilitate the reassembly of the detector protein molecule in the presence of PCR products, allowing for a real-time method for immunoPCR in vitro. Also, in another embodiment, the nucleic acid binding components of the detector molecule can facilitate the reassembly of the split-detector molecule, and therefore signal, in the presence of nucleic acids in immunoRCA (rolling circle amplification) methods, resulting in high signal amplification in vitro.

In RCA techniques a primer sequence having a region complementary to an amplification target circle (ATC) is combined with an ATC. Following hybridization, enzyme, dNTPs, etc. allow extension of the primer along the ATC template, with DNA polymerase displacing the earlier segment, generating a single stranded DNA product which consists of repeated tandem units of the original ATC sequence.

RCA techniques are well known in the art, including linear RCA (LRCA). Any such RCA technique can be used in the present invention. Strand displacement during RCA can be facilitated through the use of a strand displacement factor, such as helicase. In general, any DNA polymerase that can perform rolling circle replication in the presence of a strand displacement factor is suitable for use in the processes of the present invention, even if the DNA polymerase does not perform rolling circle replication in the absence of such a factor. Strand displacement factors useful in RCA include BMRF1 polymerase accessory subunit (Tsurumi et al., J. Virology 67(12):7648-7653 (1993)), adenovirus DNA-binding protein (Zijderveld and van der Vliet, J. Virology 68(2):1158-1164 (1994)), herpes simplex viral protein ICP8 (Boehmer and Lehman, J. Virology 67(2):711-715 (1993); Skaliter and Lehman, Proc. Natl. Acad. Sci. USA 91(22):10665-10669 (1994)), single-stranded DNA binding proteins (SSB; Rigler and Romano, J. Biol. Chem. 270:8910-8919 (1995)), and calf thymus helicase (Siegel et al., J Biol. Chem. 267:13629-13635 (1992)). The ability of a polymerase to carry out rolling circle replication can be determined by using the polymerase in a rolling circle replication assay such as those described in Fire and Xu, Proc. Natl. Acad. Sci. USA 92:4641-4645 (1995) and in Lizardi (U.S. Pat. No. 5,854,033, e.g., Example 1 therein).

Binding Motifs that Bind Non-Nucleic Acid Analytes

In some embodiments, the split-polypeptide molecule can comprise binding motifs that bind non-nucleic acid analytes. Such a motif can be, for example, a polypeptide or peptide. In other embodiments, a non-nucleic acid analyte binding motif can a biomolecule, organic molecule or inorganic molecule. In such an embodiment, the target analyte can be any metabolite, biomolecule, organic or inorganic molecule. Identification of these are known by persons or ordinary skill in the art and

### Applications.

In one embodiment of the present invention, the split-polypeptide molecule and/or split-fluorescence protein molecule produced herein can be used for real-time in vitro detection assays and for real-time detection of biomolecular interactions, such as but not limited to, detection of viral nucleic acids and/or genomes, nucleic acid detection (RNA, DNA etc); nucleic acid hybridization, such as nucleic acid duplex and triplex formation, including homo- (DNA-DNA; RNA-RNA) and hetero- (DNA-RNA etc) nucleic acid interactions. In alternative embodiments, the split-polypeptide molecule of the invention can be used for real-time in vitro detection of non-nucleic acid analytes and for the real time detection of non-nucleic acid interactions, for example biomolecules, organic molecules and inorganic molecules. In some embodiments the method of the invention can be used for detection of pathogenic and/or viral biomolecules, inorganic and organic pathogenic and/or viral molecules.

In such embodiments, the present invention is directed to methods for the real-time protein complementation. In particular, the methods of the invention are directed to real-time detection of target nucleic acid molecules, including DNA and RNA targets, as well as nucleic acid analogues. In such methods, a target nucleic acid is detected by its binding of nucleic acid binding moieties which are associated with activated split-polypeptides, wherein the binding nucleic acid binding moieties to the target nucleic acid brings the activated split-polypeptides in close proximity and immediate formation of the active protein.

In one embodiment, the nucleic acid binding moieties associated to the activated split-polypeptide fragments bind to two adjacent sequences on the target nucleic acid. In this embodiment, the adjacent sequences are close enough to each other to allow the association activated split-polypeptide fragments and assembly of the active protein when each associated nucleic acid binding moieties bound to the target nucleic acid. This embodiment provides for detection of single-stranded and double-stranded target nucleic acids. For detection of double stranded targets, the single-stranded probes interact with the double-stranded target to form a triplex.

In another embodiment, the nucleic acid binding moieties associated to the activated split-polypeptide fragments are nucleic acids or oligonucleotides and bind to the same sequence on a single-stranded target nucleic acid, forming a triplex. In this embodiment, the activated split-polypeptide fragments interact when their associated nucleic acid binding moieties are bound to the target, allowing assembly of the complementation complex.

For example, the present invention is directed to methods for the real-time protein complementation. In particular, the methods of the invention are directed to real-time detection of target analytes, including biomolecules, organic molecules and inorganic molecules, as well as fragments or metabolites thereof. In such methods, a target analyte is detected by its analyte binding motifs which are associated with activated split-polypeptides, wherein the binding of the motifs to the target analyte brings the activated split-polypeptides in close proximity and immediate formation of the active protein.

In a particular embodiment, the methods of the present invention can be used to detect the presence of a target nucleic acid of interest in vitro. Because the methods, kits and compositions of this invention are directed to the specific detection of target nucleic acids and target analytes, even in the presence of non-target molecules, they are particularly well suited for the development of sensitive and reliable probe-based hybridization assays designed to analyze for point mutations, or reliable detection of target analytes. The methods, kits and compositions of this invention are also useful for the detection, quantitation or analysis of organisms (micro-organisms), viruses, fungi and genetically based clinical conditions of interest.

In one embodiment, the present invention provides methods for isolating a target nucleic acid in a sample, even in the presence of non-target sequences. In an alternative embodiment, the invention provides for methods for isolating a target analyte in a sample.

Another important aspect of the invention is the use of the activated split-polypeptide for real-time assessment of nucleic acid hybridization and for assaying nucleic acid interactions. In such an embodiment, the present invention provides methods for real-time immediate detection of hybridization of the oligonucleotides that serve as nucleotide binding moieties conjugated to the activated split-polypeptide protein fragments. For example, localized heating (as described in Hamad-Schifferli et al., Nature, vol. 415, 10 Jan. 2002, herein incorporated by reference in its entirety) may be used to denature the bound oligonucleotides, thus dissociating the activated split-polypeptide fragments and shutting off signal and/or fluorescence. The activated split-polypeptides of the present invention are unique in that upon disassociation of the oligonucleotides, the active protein immediately disassembles and signal is ameliorated. In embodiments where the split-polypeptide fragments are split-fluorescence fragments, the fluorescence is immediately quenched or ameliorated in real-time with nucleic acid hybridization. Furthermore, the split-polypeptides are also unique in that if allowed to re-associate facilitated by hybridization of the oligonucleotides, the active protein signal (for example fluorescence) is immediately re-established.

The use of the present molecule in this embodiment allows for one to efficiently conduct and record results from various assays where multiple on-off cycling is required and allows for real time optical visualization of nucleic acid hybridization events. Further, the methods of the invention enable screening of agents which interrupt or promote hybridization and/or interfere with nucleic acid hybridization cycling events. For example, the use of activated split-polypeptide protein molecule and/or activated split-fluorescent protein molecules of this invention can be used for rapid real-time screening of agents which interfere with hybridization or hybridization cycling events. As a non-limiting or example, the methods of this invention can be used to rapidly screen for specific inhibitory nucleic acid sequences, such as antisense nucleic acids, RNAi, siRNA, shRNA, mRNAi etc, and/or agents which promote or prevent the activity of such inhibitory nucleic acids. In such an embodiment, agents or molecules that decrease hybridization between the binding moieties associated with the activated split-fluorescent protein results in an attenuated or decreased active protein signal, whereas agents promoting hybridization between the binding moieties result in increased active protein signal.

In another embodiment, the molecule can be used for real-time quantification of nucleic acids. In related embodiment, the methods of the present invention can be used for immunoRCA and immuno PCR methods. In another embodiment of the invention provides for the use of the real-time protein complementation to screen for a target nucleic acid in vitro. For example, to identify a target nucleic acid of interest in a population of other non-target nucleic acids. In this embodiment, the target nucleic acids or the split-polypeptide molecule of the present invention can be used in a form in which they are attached, by whatever means is convenient, to some type of solid support. Attachment to such supports can be by means of some molecular species, such as some type of polymer, biological or otherwise, that serves to attach said primer or ATC to a solid support so as to facilitate detection of tandem sequence DNA produced by rolling circle amplification using the methods of the invention.

Such solid-state substrates useful in the methods of the invention can include any solid material to which oligonucleotides can be coupled. This includes materials such as acrylamide, cellulose, nitrocellulose, glass, polystyrene, polyethylene vinyl acetate, polypropylene, polymethacrylate, polyethylene, polyethylene oxide, glass, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Solid-state substrates can have any useful form including thin films or membranes, beads, bottles, dishes, fibers, woven fibers, shaped polymers, particles and microparticles. A preferred form for a solid-state substrate is a glass slide or a microtiter dish (for example, the standard 96-well dish). For additional arrangements, see those described in U.S. Pat. No. 5,854,033.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994). A preferred method of attaching oligonucleotides to solid-state substrates is described by Guo et al., Nucleic Acids Res. 22:5456-5465 (1994).

In another embodiment, the molecule of the invention can be used for quantification of non-nucleic acid analytes. In another embodiment of the invention provides for the use of the real-time protein complementation to screen for a target analytes in vitro. For example, to identify a target analyte of interest in a population of other non-target analytes. In this embodiment, the binding motif of the analyte conjugated to the split-polypeptide molecule of the present invention can be used in a form in which they are attached, by whatever means is convenient, to some type of solid support. Attachment to such supports can be by means of some molecular species, such as some type of polymer, biological or otherwise, that serves to attach said primer or ATC to a solid support so as to facilitate detection of the analyte DNA produced by rolling circle amplification using the methods of the invention.

Another important embodiment of the present invention is use of the split-polypeptide molecule for real-time detection of specific nucleic acid sequences in vitro. In particular the present invention allows for the real-time detection of gene mutations, polymorphisms, or aberrations in an individual. A biological sample is isolated from an individual and DNA and/or RNA is extracted. The molecule of the present invention is designed so that the split fluorescent protein is bound to oligonucleotides that are specific for the particular mutation, polymorphism or aberration one is trying to detect. Alternatively, a pool of molecules may be used whereby many mutations, polymorphisms, or aberrations may be detected. In this embodiment, the oligonucleotides attached to the split fluorescent proteins are complementary for each other and thus the baseline is fluorescence. The individual DNA and/or RNA is then contacted to said molecule(s). If the individual has the particular mutation or polymorphism, it will compete with the split fluorescent molecule and reduce fluorescence. Preferably, the individual's DNA and/or RNA is amplified prior to contact with the fluorescent molecule. This is particularly useful in the detection of single nucleotide polymorphisms of know polymorphisms. The present molecule allows for sensitive detections due to the immediacy of fluorescent detection

In one embodiment, the molecule can be used for real-time detection of pathogens in vitro. In one embodiment, the molecule of the invention can be used to detect the presence of pathogen nucleic acid sequences and/or aberration in nucleic acid sequences as a result of presence of pathogen and/or pathogen nucleic acid. In alternative embodiments, the molecule of the invention can be used to detect the presence of an non-nucleic acid analyte as a result of infection with a pathogen. The pathogen can be a virus infection, fungi infection, bacterial infection, parasitic infection and other infectious diseases. Viruses can be selected from a group of viruses comprising of Herpes simplex virus type-1, Herpes simplex virus type-2, Cytomegalovirus, Epstein-Barr virus, Varicella-zoster virus, Human herpes virus 6, Human herpes virus 7, Human herpes virus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B. Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B. Rotavirus C, Sindbis virus, Simian hnmunodeficiency cirus, Human T-cell Leukemia virus type-1, Hantavirus, Rubella virus, Simian Enmunodeficiency virus, Human Immunodeficiency virus type-1, and Human Immunodeficiency virus type-2.

Detection of target nucleic acid or target analytes may also be useful for the detection of bacteria and eukaryotes in food, beverages, water, pharmaceutical products, personal care products, dairy products or environmental samples. Preferred beverages include soda, bottled water, fruit juice, beer, wine or liquor products. Assays developed will be particularly useful for the analysis of raw materials, equipment, products or processes used to manufacture or store food, beverages, water, pharmaceutical products, personal care products, dairy products or environmental samples.

In another related embodiment of the invention, the assembly of the activated split-fluorescent polypeptides form an assembled protein which contains a discontinuous epitope, which may be detected by use of an antibody which specifically recognizes the discontinuous epitope on the assembled protein but not the partial epitope present on either individual polypeptide. One such example of a discontinuous epitope is found in gp120 of HIV. These antigens can be use as detector proteins for subsequent detection by methods known in the art, such as immunodetection. These and other such derivatives can readily be made by the person of ordinary skill in the art based upon well known techniques, and screened for antibodies that recognize the assembled protein by neither protein fragment on its own.

The target nucleic acid can be of human origin. The target nucleic acid can be DNA or RNA. The target nucleic acid can be free in solution or immobilized to a solid support.

In one embodiment, the target nucleic acid or target analyte is specific for a genetically based disease or is specific for a predisposition to a genetically based disease. Said diseases can be, for example, .beta.-Thalassemia, Sickle cell anemia or Factor-V Leiden, genetically-based diseases like cystic fibrosis (CF), cancer related targets like p53 and p10, or BRC-1 and BRC-2 for breast cancer susceptibility. In yet another embodiment, isolated chromosomal DNA may be investigated in relation to paternity testing, identity confirmation or crime investigation.

The target nucleic acid or target analyte can be specific for a pathogen or a microorganism. Alternatively, the target nucleic acid or target analyte can be from a virus, bacterium, fungus, parasite or a yeast; wherein hybridization of the complementation molecules to the target nucleic acid is indicative of the presence of said pathogen or microorganism in the sample.

In another embodiment, the present invention provides kits suitable for detecting the presence and/or amount of a target nucleic acid or target analyte in a sample. The kits comprise at least a first probe coupled to a first molecule and a second probe coupled to a second molecule, wherein the probes can bind to a hybridization sequence in a target nucleic acid. Preferably, the probes are in vials. The kits also comprise reagents suitable for capturing and/or detecting the present or amount of target nucleic acid or target analyte in a sample. The reagents for detecting the present and/or amount of target nucleic acid and or target analyte can include enzymatic activity reagents or an antibody specific for the assembled protein. The antibody can be labeled. Such kits may optionally include the reagents required for performing RCA reactions, such as DNA polymerase, DNA polymerase cofactors, and deoxyribonucleotide-5′-triphosphates. Optionally, the kit may also include various polynucleotide molecules, DNA or RNA ligases, restriction endonucleases, reverse transcriptases, terminal transferases, various buffers and reagents, and antibodies that inhibit DNA polymerase activity. These components are in containers, such as vials. The kits may also include reagents necessary for performing positive and negative control reactions, as well as instructions. Optimal amounts of reagents to be used in a given reaction can be readily determined by the skilled artisan having the benefit of the current disclosure.

In another embodiment, the methods of the invention can be used for protein complementation for multiple nucleic acid targets or multiple analytes simultaneously. As an exemplary non-limiting example, protein complementation of complementary split-polypeptide fragments which have associated different nucleic acid binding motifs. For example, the presence of one target nucleic acid will facilitate protein complementation of one active split-polypeptide fragment pair, while the presence of another target will facilitate protein complementation of anther pair of activated split-polypeptide fragments, resulting in a different active protein and detectable signal. In such an embodiment, multiple nucleic acid targets can be detected simultaneously. In an alternative embodiment, simultaneous detection of target nucleic acids, such as RNA and DNA can be monitored by real-time protein complementation. In an alternative embodiment, the multiple non-nucleic acid analytes can be detected simultaneously by use of a split-polypeptide fragment comprising specific analyte binding motifs. Such an embodiment would be particularly useful, for example, in assessing the presence or the level of more than one analyte which contribute to the symptoms of for the diagnosis of a disease, disorder or dysfunction.

In a related embodiment, the multiple protein complementation using split-fluorescent protein fragments from different fluorescent proteins. In a related embodiment, the methods of the invention enable real-time detection and identification of specific target nucleic among a variety of other putative but different nucleic acid targets (see Hu et al, Nature Biotechnology, 2003; 21; 539-545; Kerppola, 2006, 7; 449-456, Hu, et al, Protein-Protein Interactions (Ed. P. Adams and E. Golemis), Cold Spring Harbor Laboratory Press. 2005, herein incorporated by reference in its entirety).

### DEFINITIONS

Unless stated otherwise, the following terms and phrases as used herein are intended to have the following meanings:

The term “refolding” refers to the folding of the dissociated protein molecules produced in the solubilizing process into their native three-dimensional conformation. This procedure is affected by the amino acid sequence of the protein. It is well-known that the disulfide bonds are formed in correct positions when the refolding precedes the formation of disulfide bonds in a protein, thereby causing the formation of an active protein of native conformation.

The term “preformed” as used herein refers to an already formed conformation and structure. The term “preformed chromophore” refers to the mature conformation of the chromophore that is necessary for production of fluorescence. A preformed chromophore is in the active conformation and does not need structural modification to become active.

The term “polynucleotide” refers to any one or more nucleic acid segments, or nucleic acid molecules, e.g., DNA or RNA fragments, present in a nucleic acid or construct. A “polynucleotide encoding an gene of interest” refers to a polynucleotide which comprises the coding region for such a polypeptide. In addition, a polynucleotide may encode a regulatory element such as a promoter or a transcription terminator, or may encode a specific element of a polypeptide or protein, such as a secretory signal peptide or a functional domain.

A “nucleotide” is a monomer unit in a polymeric nucleic acid, such as DNA or RNA, and is composed of three distinct subparts or moieties: sugar, phosphate, and nucleobase (Blackburn, M., 1996). When part of a duplex, nucleotides are also referred to as “base” or “base pairs”. The most common naturally-occurring nucleobases, adenine (A), guanine (G), uracil (U), cytosine (C), and thymine (T) bear the hydrogen-bonding functionality that binds one nucleic acid strand to another in a sequence specific manner. “Nucleoside” refers to a nucleotide that lacks a phosphate. In DNA and RNA, the nucleoside monomers are linked by phosphodiester linkages, where as used herein, the term “phosphodiester linkage” refers to phosphodiester bonds or bonds including phosphate analogs thereof, including associated counter-ions, e.g., IT′, NW, Na′, and the like.

As used herein, the terms “oligonucleotide” and “primer” have the conventional meaning associated with it in standard nucleic acid procedures, i.e., an oligonucleotide that can hybridize to a polynucleotide template and act as a point of initiation for the synthesis of a primer extension product that is complementary to the template strand.

“Polynucleotide” or “oligonucleotide” refer to linear polymers of natural nucleotide monomers or analogs thereof, including double and single stranded deoxyribonucleotides “DNA”, ribonucleotides “RNA”, and the like. In other words, an “oligonucleotide” is a chain of deoxyribonucleotides or ribonucleotides, that are the structural units that comprise deoxyribonucleic acid (DNA) and ribonucleic acid (RNA), respectively. Polynucleotides typically range in size from a few monomeric units, e.g. 8-40, to several thousand monomeric units. Whenever a DNA polynucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, unless otherwise noted.

“Watson/Crick base-pairing” and “Watson/Crick complementarity” refer to the pattern of specific pairs of nucleotides, and analogs thereof, that bind together through hydrogen-bonds, e.g. A pairs with T and U, and G pairs with C. The act of specific base-pairing is “hybridization” or “hybridizing”. A hybrid forms when two, or more, complementary strands of nucleic acids or nucleic acid analogs undergo base-pairing.

As used herein, the terms “oligonucleotide” and “primer” have the conventional meaning associated with it in standard nucleic acid procedures, i.e., an oligonucleotide that can hybridize to a polynucleotide template and act as a point of initiation for the synthesis of a primer extension product that is complementary to the template strand.

Many of the oligonucleotides described herein are designed to be complementary to certain portions of other oligonucleotides or nucleic acids such that stable hybrids can be formed between them. The stability of these hybrids can be calculated using known methods such as those described in Lesnick and Freier, Biochemistry 34:10807-10815 (1995), McGraw et al., Biotechniques 8:674-678 (1990), and Rychlik et al., Nucleic Acids Res. 18:6409-6412 (1990).

“Conjugate” or “conjugated” refer to the joining of two or more entities. The joining can be fusion of the two or more polypeptides, or covalent, ionic, or hydrophobic interactions whereby the moieties of a molecule are held together and preserved in proximity. The attachment of the entities may be together by linkers, chemical modification, peptide linkers, chemical linkers, covalent or non-covalent bonds, or protein fusion or by any means known to one skilled in the art. The joining may be permanent or reversible. In some embodiments, several linkers may be included in order to take advantage of desired properties of each linker and each protein in the conjugate. Flexible linkers and linkers that increase the solubility of the conjugates are contemplated for use alone or with other linkers are incorporated herein. Peptide linkers may be linked by expressing DNA encoding the linker to one or more proteins in the conjugate. Linkers may be acid cleavable, photocleavable and heat sensitive linkers.

The term “moieties” or “motif” used interchangeably herein, refers to a molecule; nucleic acid or protein or otherwise, capable of performing a particular function. “Nucleic acid binding moieties” or “nucleic acid binding motif” refers to an molecule capable of binding to the nucleic acid in specific manner.

“Detection” refers to detecting, observing, or measuring a construct on the basis of the properties of a detection label.

The term “nucleobase-modified” refers to base-pairing derivatives of AGC, T, U, the naturally occurring nucleobases found in DNA and RNA.

The term “promoter” refers to the minimal nucleotide sequence sufficient to direct transcription. Also included in the invention are those promoter elements that are sufficient to render promoter-dependent gene expression controllable for cell-type specific, tissue specific, or inducible by external signals or agents; such elements may be located in the 5′ or 3′ regions of the native gene, or in the introns. The term “inducible promoter” refers to a promoter where the rate of RNA polymerase binding and initiation of transcription can be modulated by external stimuli. The term “constitutive promoter” refers to a promoter where the rate of RNA polymerase binding and initiation of transcription is constant and relatively independent of external stimuli. A “temporally regulated promoter” is a promoter where the rate of RNA polymerase binding and initiation of transcription is modulated at a specific time during development. All of these promoter types are encompassed in the present invention.

The term “polypeptide” or “peptide” are used interchangeably herein refer to a protein.

The term “in vitro” as used herein is intended to encompass any solution or any cell that is outside the organism. Typically, in vitro refers to reactions occurring in a test tube, vial or any other container or holder, where the solution and/or cell is separated from the environment from which it is normally found.

The term “analyte” as used in the context of non-nucleic acid analyte herein, is intended to refer to any chemical, biological or structural entity that is not a nucleic acid or nucleotide or nucleic acid analogue. Such an analyte includes, but is not limited to organic molecules, inorganic molecules, biomolecules, metabolites etc.

### EXAMPLES

### Example 1

### Methods

Molecular modeling. Modeling of EGFP and its fragments was performed using a string of beads method¹⁸. Each amino acid of a polypeptide is represented by two beads corresponding to the C_{α} and C_{β} positions. Neighboring beads are constrained to mimic the backbone geometry and flexibility. The interactions between amino acids are simulated by a Gō-like structure-based potential¹⁸. In such a model, two amino acids are assigned an attractive or repulsive potential depending on whether they form a contact in the native protein state or not. The conformation of native EGFP was taken from the Protein Database Bank (X-ray structure; PDB code 1c4f). To choose the contact potential for amino acids in EGFP fragments we used native structures of a full-size protein. Protein folding thermodynamics and kinetics were analyzed by the discrete molecular dynamics (DMD) approach¹⁸.

Cloning, expression and purification of polypeptides. A plasmid containing EGFP-1 gene (Clontech) was used as a template for PCR amplification of DNA sequences coding for the large (A) and small (B) EGFP fragments. The large fragment contained 158 N-terminal amino acids plus a C-terminal cysteine and the small fragment contained remaining C-terminal 81 amino acids plus an N-terminal cysteine. PCR products were cloned in the TWIN-1 vector (New England Biolabs) to yield the C-terminal fusions of Ssp DNAB intein (to purify the desired protein fragments using the intein self-splitting chemistry^{21,22}), and expressed in BL21(DE3) pLys competent E. coli cells (Stratagene). The structure of all constructs was verified by sequencing. Primers for PCR amplification are: Large EGFP fragment with C-terminal cysteine: Primer ALPHA_dir: 5′-AGTTTCTAGAATGGTGAGCAAGGGCG (SEQ ID NO.1); Primer ALPHA-CYS_rev: 5′-ATCGCTCGAGTTAGCACTGCTTGTCGGCCATG (SEQ ID NO.2); biotinylated oligo 1: biotin-5′-CGACTGCGTTAGCATGTGTTG (SEQ ID NO.3). Small EGFP fragment with N-terminal cysteine: Primer BETA-CYS_dir: 5′-ATCGGATATCATGTGCAAGAACGGCATCAAGGTG (SEQ ID NO.4); Primer BETA_rev: 5′-ATCGCTCGAGTTACTTGTACAGCTCGTCC (SEQ ID NO.5); biotinylated oligo 2: 5′-CAACACATGCTAACGCAGTCG-biotin (SEQ ID NO.6).

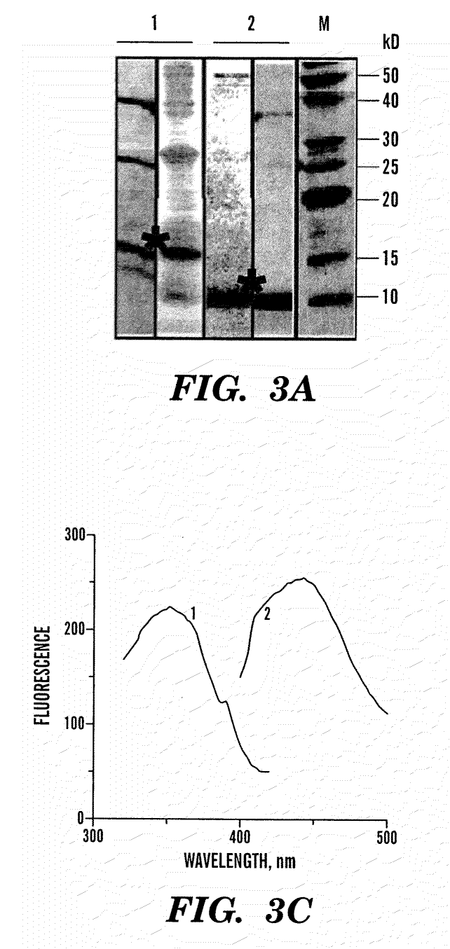

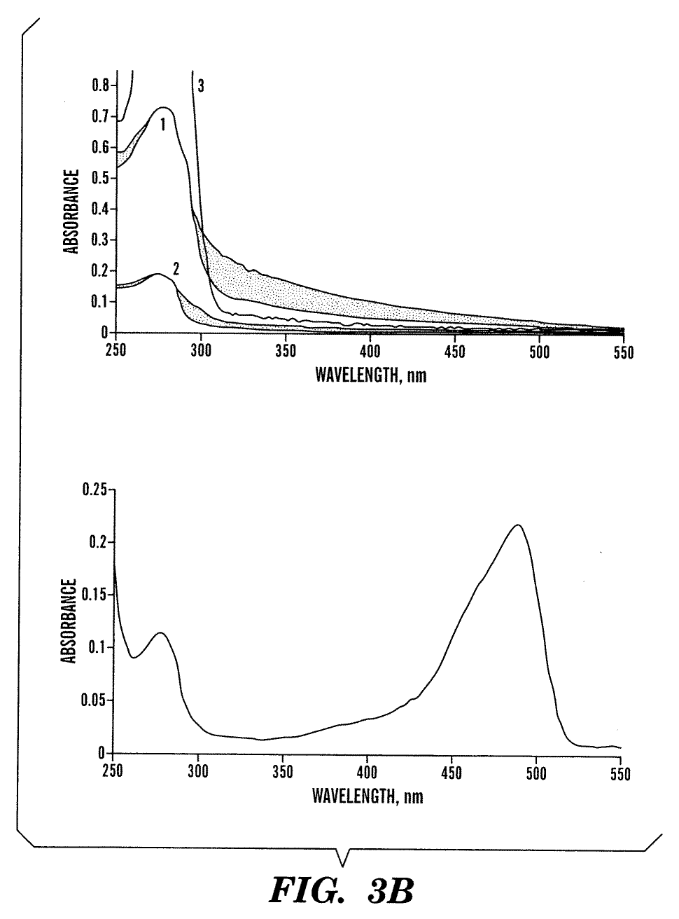

Cells were grown overnight to OD₆₀₀=0.6 and induced with 0.35 mM IPTG overnight at 25° C. Cells were pelleted by centrifugation, washed with a buffer containing 50 mM Tris-HCl, pH 8.5, 25% sucrose, 1 mM EDTA, 10 mM DTT) and frozen (−70° C. for 10 min) and thawed (37° C. for 5 min) 3 times. Cells were lysed by sonication with 3×30 sec bursts each followed by 30 sec intervals when the cells were kept on ice (Sonifier cell disrupter W185c, Branson Sonic Power). The resulting mixture was centrifuged at 15000 rpm for 5 min at 4° C., the pellet resuspended in the same buffer and sonicated again for additional 3×30 sec bursts. The pellet was washed 3 times and then resuspended in the buffer containing 25 mM MES pH 8.5, 8 M urea, 10 mM NaEDTA, 0.1 mM DTT and left at room temperature for 1 hr. The solubilized proteins were centrifuged at 15000 rpm for 5 min and the supernatant was then refolded by adding drop by drop to the refolding buffer (50 mM Tris pH 8.5, 500 mM NaCl, 1 mM DTT) with dilution ratio of 1:100. The refolded proteins were purified using chitin columns as recommended by the supplier. The purity of all proteins was analyzed by SDS-PAGE (FIG. 3A). Protein absorption spectra were recorded on a Hitachi U-3010 spectrophotometer.

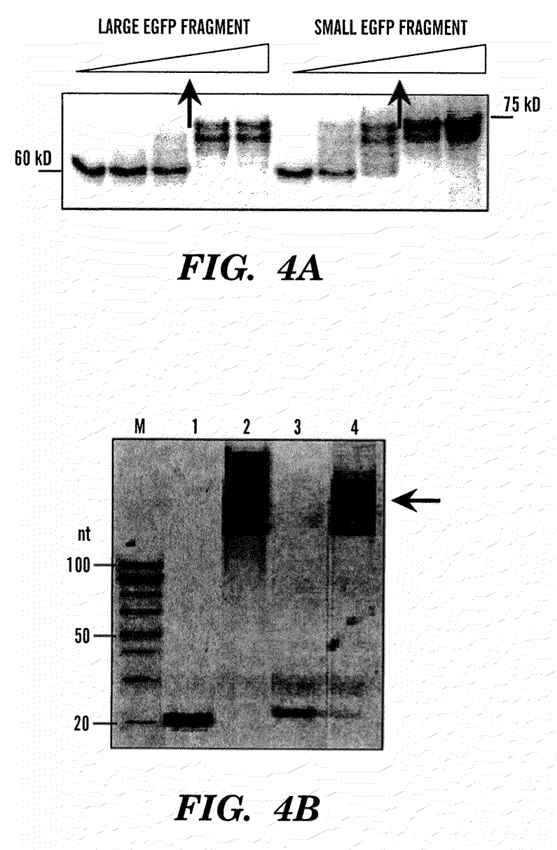

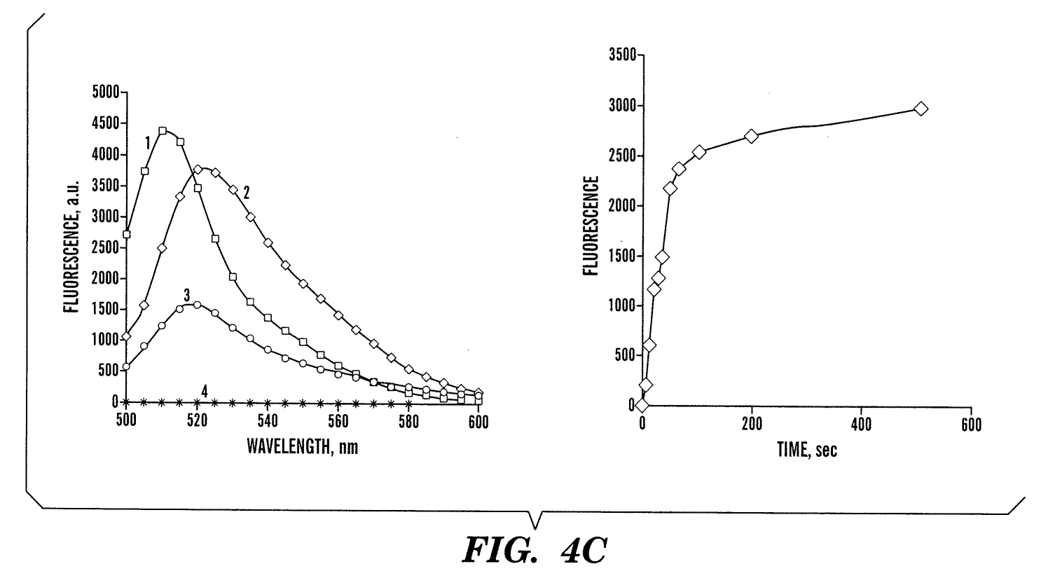

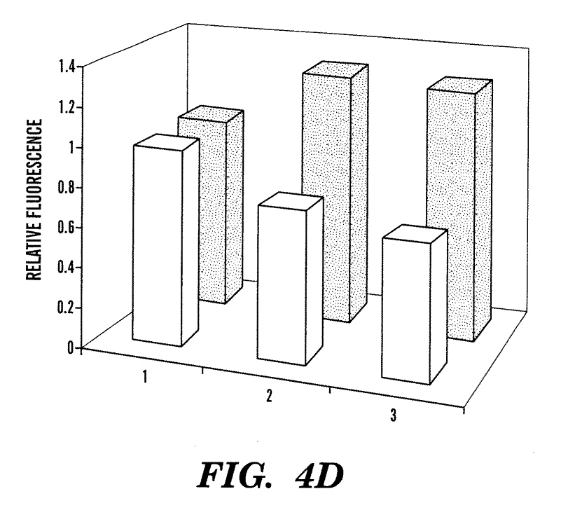

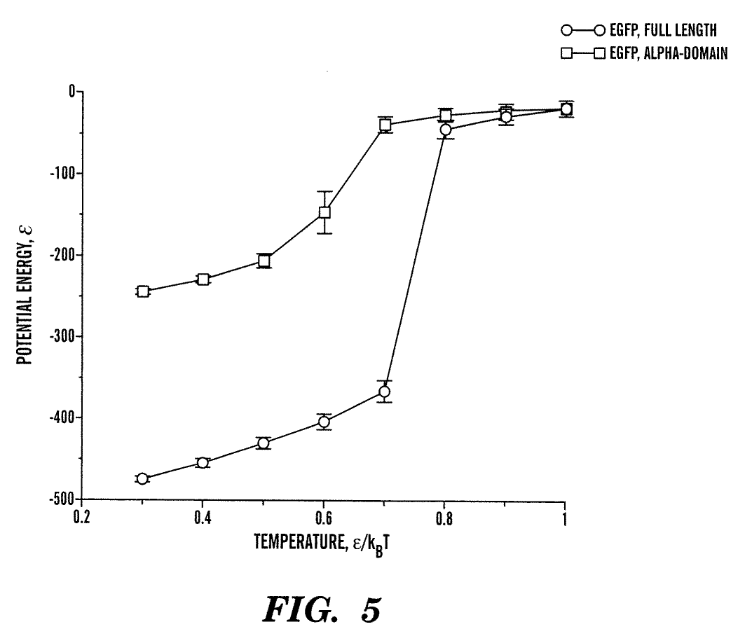

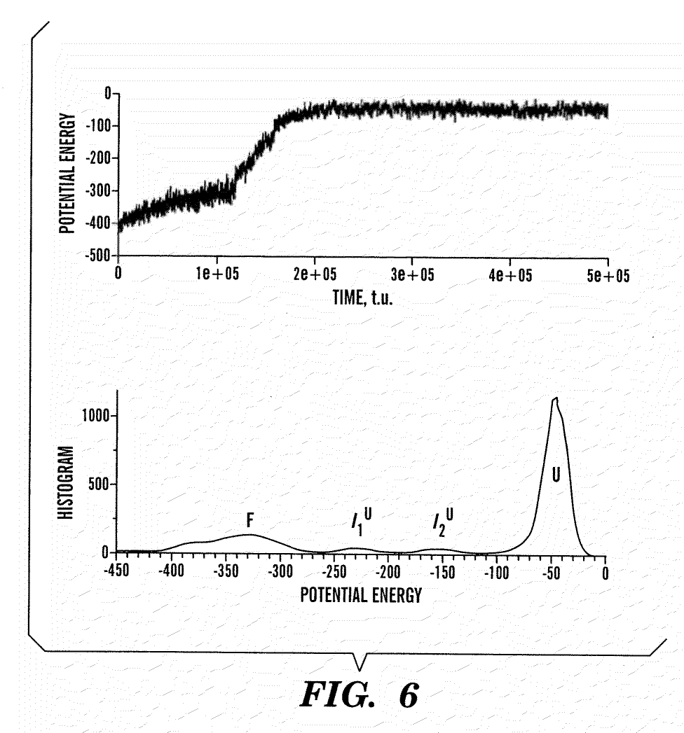

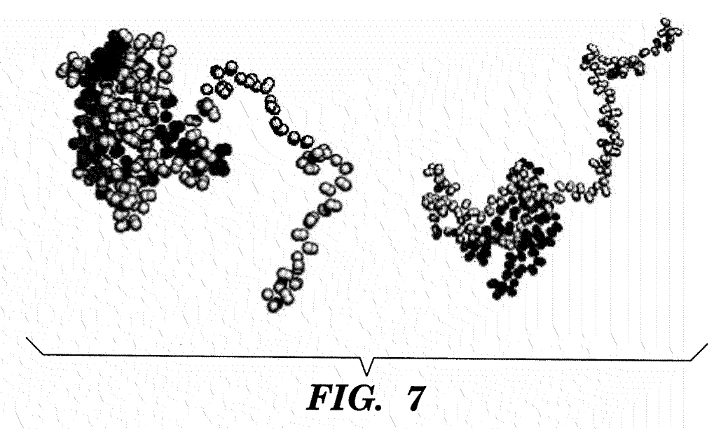

Coupling of proteins with oligonucleotides, protein complementation and fluorescence measurements. The EGFP protein fragments were first gel-filtrated into the PBS-EDTA buffer, pH 7.5 using G-25 microspin columns (Amersham Biosciences). Then, these Solutions were mixed at 10:1 volume ratio with 10 mM biotin-HPDP (Pierce) in dimethylformamide and incubated 2 hr at room temperature to reach ≧70% biotinylation. Unreacted biotin-HPDP was removed from biotinylated proteins by gel filtration. Next, 1:1 complexes of biotinylated EGFP fragments with streptavidin were obtained by incubating these fragments with equimolar amounts of streptavidin (as determined by titration experiments; see FIG. 4A) for 15 min at 37° C. in PBS-EDTA buffer. Finally, an equimolar amount of the corresponding biotinylated oligonucleotide has been added to each binary complex to get 1:1:1 tripartite molecular constructions. The tripartite molecular constructions thus obtained (see FIG. 4B) were mixed at 1:1 molar ratio in the PBS-EDTA buffer to final concentration ˜200 nM. Fluorescence responses of the restored, split EGFP were monitored on a Hitachi F-2500 spectrofluorometer. To dissociate the restored oligonucleotide-supported protein constructs, a hundred-fold excess of the non-biotinylated oligonucleotide (with the same sequence as biotinylated oligomer used for coupling with the large EGFP fragment) was added, and the resulting fluorescence changes were recorded.

### Results

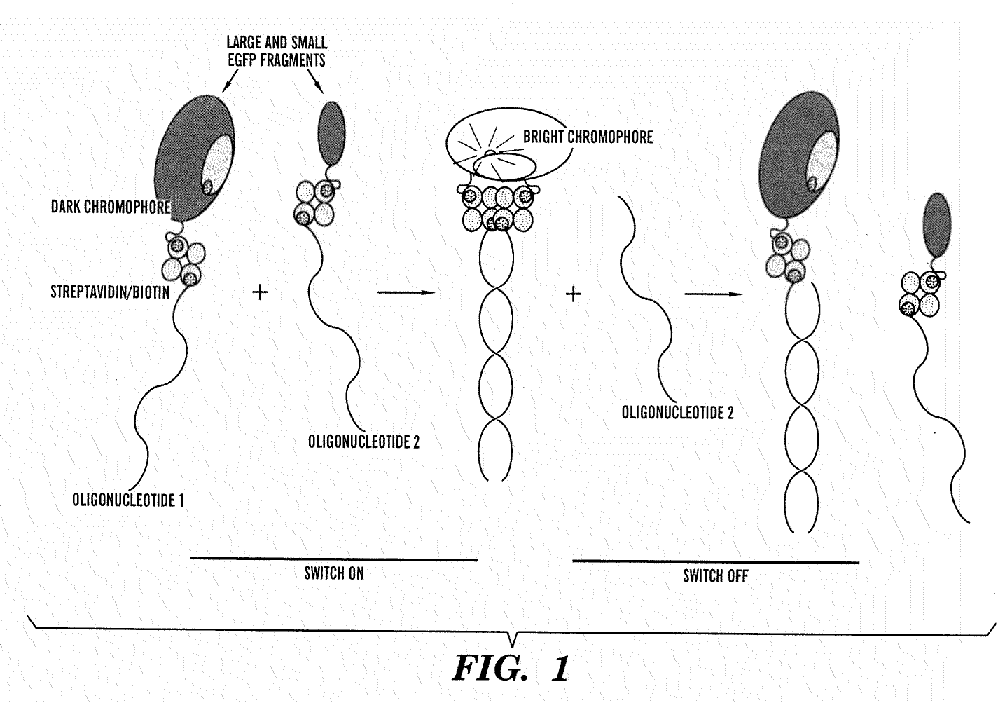

In our design (FIG. 1A), two fragments of a fluorescent protein are coupled with complementary oligonucleotides. One polypeptide contains a chromophore that is capable of bright fluorescence within a full-size protein. However, this chromophore is not fluorescent in a protein fragment because it is exposed to and quenched by solvent, and it may also lack necessary contacts with amino acids of the other fragment. When the two protein fragments are brought close to each other by nucleic acid complementary interactions, the second polypeptide acts as a shield for the chromophore isolating it from solution and allowing restoration of all missing amino acid contacts which results in development of fluorescence. In this study, we manipulated two fragments of the enhanced green fluorescent protein (EGFP)², which correspond to its large and small domains linked by a flexible loop of nine amino acids (153-161 EGFP residues)¹⁶. Larger, N-terminal EGFP domain is known to contain three amino acids forming a chromophore that fluoresces in native but not in denatured protein^{2,17}. Moreover, this tripeptide exhibits no fluorescence in a separate large EGFP fragment⁶⁻⁹.

The EGFP chromophore formation is a self-catalytic process requiring correct protein folding¹⁷. We hypothesized that the N-terminal EGFP fragment (˜⅔ of the entire EGFP) was large enough to develop a compact folded structure by itself. We also hypothesized that the structure would be so conformationally close to a corresponding part of the complete EGFP that the chromophore would be spontaneously formed within the folded large EGFP fragment, even though it is not fluorescent.

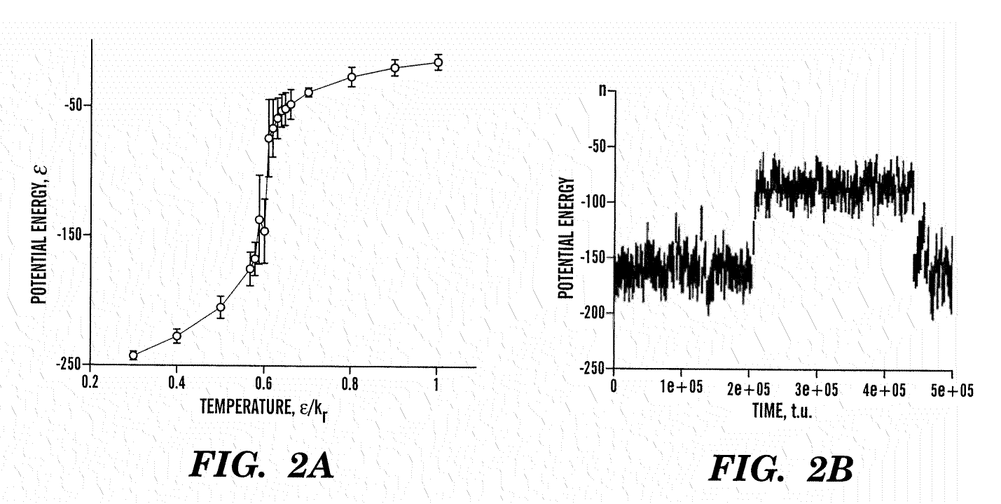

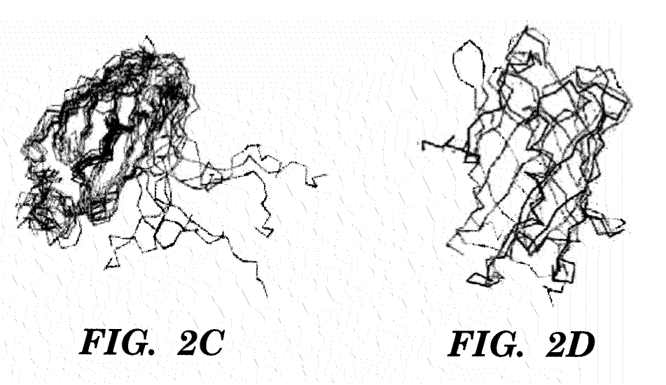

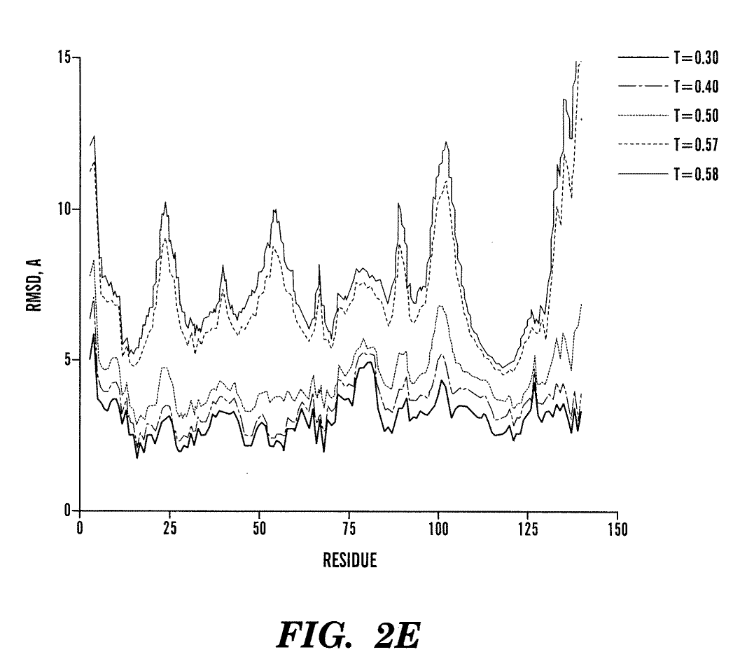

We performed molecular modeling analyses of the EGFP and its large fragment using discrete molecular dynamics (DMD) simulations¹⁸. The results of DMD simulations are temperatures (T<0.6) the large EGFP fragment is indeed folded featuring a substantially decreased potential energy; at higher temperatures (T>0.6) the protein remains unfolded with a high potential energy. Folding thermodynamics and kinetics of this polypeptide follow a two-state, all-or-none mode typical for single-domain proteins. Near the transition temperature T_{F}˜0.60, the large EGFP fragment displays both the folded and unfolded states with approximately equal probability, and with large fluctuations in potential energy (FIG. 2B). During the folding and unfolding events, no intermediate states of the large EGFP fragment were observed.

FIG. 2C demonstrates a compact structure of the folded large EGFP fragment, except for its dangling 20-residues-long C-terminal part. Moreover, the arrangements of the chromophore-forming amino acids in the full-size EGFP and within its large fragment are essentially the same (FIG. 2D, E), hence making possible the chromophore formation. Still, FIG. 2C shows that the chromophore-forming amino acids in the large EGFP fragment are exposed to a solvent, which is not the case in the full-size EGFP, where these amino acids are buried deep inside the protein (FIG. 2D). Besides, these amino acids lack many important contacts with other residues of the smaller EGFP fragment, which are present in the full-size protein¹⁹. Thus, even if the chromophore formed, it might be deficient in exhibiting strong fluorescence when within incomplete EGFP.

The small EGFP fragment consists of two β-hairpins, which do not contact each other, so that this polypeptide cannot form a well-defined compact structure by itself. However, our DMD simulations of the EGFP folding suggest that once the small EGFP fragment binds to its larger counterpart, it finds the correct position to become a part of the united compact protein structure, and the dangling part in the large EGFP fragment also folds consequently.

We then genetically dissected EGFP between amino acids 158 and 159 within a flexible loop by cloning and isolating two separate fragments of this protein that correspond to the large and small domains used for the DMD simulations. For optimal functionality, the split EGFP-based optical switch should be able to quickly respond to the DNA hybridization-dehybridization events. Nucleic acid complementary interactions are known to be fast (within minutes)^{12,13,20}. In contrast, de novo formation of the mature pro-fluorescent EGFP chromophore requires hours¹⁷. Based on molecular modeling analyses we suspected that the large EGFP fragment can be isolated in vitro with the pre-formed chromophore. If this is the case, then the fluorescently-active complementation of two EGFP fragments should proceed fast and take a few minutes instead of several hours. Note that in all prior reports, EGFP re-assembly in vitro was performed most likely with the protein lacking mature chromophore, which formed only as a result of re-assembly⁶⁻⁹. Therefore, the fluorescence development in these studies was very slow.

The large and small EGFP fragments were overexpressed in E. coli as fusions with small self-splitting Ssp DNAB intein²¹ to facilitate the protein purification²². These polypeptides were isolated from inclusion bodies after refolding (see Methods for details). It has been shown that intein in fusions with fluorescent protein did not affect its proper folding²². FIG. 3A shows that both EGFP fragments were obtained with high enough purity: refolded protein samples contained ≧70% of the large and ˜90% of the small EGFP fragments.

FIG. 3B shows the absorption spectra of these polypeptides. One can see that both EGFP fragments lack a characteristic peak at 490 nm seen in native EGFP (FIG. 3B inset). However, in contrast to the small EGFP fragment and other nonfluorescent/chromophore-free protein (streptavidin), the large EGFP fragment features significant absorbance in the range 300-400 nm, which is expected for the chromophore of the denatured EGFP¹⁷ and which was also observed for other photoactive split EGFP variants²³. The presence of chromophore in the large EGFP fragment becomes more evident in the fluorescence spectra (FIG. 3C): this fragment exhibits weak fluorescence (˜100 times weaker as compared to peak fluorescence of intact EGFP) with distinct maxima at 360 nm in excitation and at 460 nm in emission spectra. These spectra are quite different from those of the full-length EGFP (see FIG. 4A for the EGFP emission spectrum; the EGFP excitation spectrum resembles its absorption spectrum shown in FIG. 3B inset). However, they correspond to fluorescence spectra of the synthetic chromophore, and to the spectra of a short, chromophore-containing peptide isolated from the intact fluorescent protein by partial proteolysis²⁴. Thus, these data indicate that the large EGFP fragment isolated and refolded from inclusion bodies contains a pre-formed chromophore.

For DNA-supported EGFP complementation, protein fragments were coupled with complementary oligonucleotides using biotin-streptavidin chemistry (FIG. 1). The large and small EGFP fragments were expressed with extra cysteine residues at the C- and N-termini, respectively, for biotinylation with the sulfhydryl-reactive reagent, biotin-HPDP. The C- and N-terminally biotinylated polypeptides can be then coupled with biotinylated oligonucleotides via streptavidin; this high-affinity biotin-binding protein^{25,26} acts as a linker. We assumed that the terminal Cys in the A fragment of EGFP will be the major target site for biotinylation, while internal Cys₄₉ and Cys₇₁, which are buried to some extent inside the polypeptide (as supported by the DMD structure in FIG. 2c, e) will be much less reactive.

We chose this non-covalent coupling because it allows modular design^{27,28}, which can be advantageous when different EGFP-based optical switches are prepared. Note that the link formed between the protein and biotin-HPDP via S—S bonding can be readily cleaved with reducing agents, if subsequent disassembly is necessary. In planning this design, we assumed that its spatial arrangement would simultaneously allow the oligonucleotides to form duplexes and the EGFP fragments to come close to each other. Indeed, when two streptavidin molecules are located side by side, their centers are separated by ˜60 Å²⁵. Given that the biotin-binding site is located near the middle of each streptavidin subunit²⁶, one can estimate the smallest distance between the two such sites in the contacting proteins as ˜30 Å. The length of biotin linkers in biotin-HPDP reagent and in the oligonucleotides was ≧25 Å, thus being sufficient for all corresponding partners of the assembly to associate.

The biotinylated EGFP fragments were attached to streptavidin at a 1:1 ratio (FIG. 4a), and then coupled with the corresponding oligonucleotides bearing biotin at the 5′- or 3′-end (FIG. 4b; see FIG. 1 for schematics). When these tripartite molecular constructions were combined in equimolar amounts, strong increase in fluorescence was detected with excitation/emission spectra resembling EGFP (FIG. 4c). In contrast, control experiments with mixing streptavidin-bound protein fragments without complementary oligonucleotides did not show any appreciable fluorescence. The kinetics of the DNA-templated EGFP re-assembly was fast with a t_{1/2}≦1 min (FIG. 4a inset). This is close to the kinetics of renaturation of EGFP from denatured protein with mature chromophore^{2,17}, and agrees well with essentially immediate formation of DNA duplexes²⁰. The fluorescent intensity of the re-assembled complexes varied from experiment to experiment with maximal response close to that of the intact EGFP.

Two differences between the fluorescence spectra of the intact EGFP and re-assembled protein should be noted. First, the excitation/emission maxima for re-assembled protein were red-shifted to 490/524 nm, as compared to 488/507 nm for EGFP. The spectral changes can be explained by somewhat different arrangement of amino acids surrounding the chromophore within the re-assembled protein as well as by the presence of streptavidin and/or negatively charged DNA within the complex. The second difference becomes apparent upon addition of Mg²⁺ ions. The fluorescence of native EGFP gradually decreases after addition of 2 mM MgSO₄ and reaches about 70% of its initial value in 3 hr after, in accordance with the known quenching effect of bivalent cations on EGFP fluorescence². In contrast, the fluorescence of the re-assembled complex increased about 30% within a few minutes upon addition of Mg²⁺ and remained essentially unchanged (FIG. 4d). This effect can be explained by a stabilizing effect of Mg²⁺ on duplex DNA, which is playing a major role in the re-assembly of EGFP within the DNA-protein complex.

Finally, we examined the possibility of turning off the fluorescence of restored split EGFP by dissociating the assembled multicomponent complex. For this purpose, we also employed DNA hybridization (see second part of FIG. 1). When one of the two complementary oligonucleotides was added in excess to the fluorescent complex, an essentially instant drop in fluorescence has been detected (FIG. 4c). Evidently, the competing hybridization of a non-tagged oligonucleotide displaces its protein-tagged equivalent and, as a result, splits the complemented protein complex. Alternatively, the DNA hybridization-dehybridization events could be remotely controlled by local heating²⁰ making it possible to perform multiple on-off cycling of optical signal generated in the system. We termed this approach Swift & Winked Illumination Triggered & Controlled by Hybridization (SWITCH) meaning its possible applications.

Aequorea victoria Green-Fluorescent Protein (Accession M62653):

(SEQ ID NO.7)

MSKGEELFTGVVPILVELDGDVNGHKFSVSGEGEGDATYGKLTLKFICTT
GKLPVPWPTLVTTFSYGVQCFSRYPDHMKQHDFFKSAMPEGYVQERTIFF
KDDGNYKTRAEVKFEGDTLVNRIELKGIDFKEDGNILGHKLEYNYNSHNV
YIMADKQKNGIKVNFKIRHNIEDGSVQLADHYQQNTPIGDGPVLLPDNHY
LSTQSALSKDPNEKRDHMVLLEFVTAAGITHGMDELYK.

Aequorea victoria Green-Fluorescent Protein mRNA, Complete cds (Accession M62653):

(SEQ ID NO.8)

tacacacgaa taaaagataa caaagatgag taaaggagaa
gaacttttca ctggagttgt cccaattctt gttgaattag
atggtgatgt taatgggcac aaattttctg tcagtggaga
gggtgaaggt gatgcaacat acggaaaact tacccttaaa
tttatttgca ctactggaaa actacctgtt ccatggccaa
cacttgtcac tactttctct tatggtgttc aatgcttttc
aagataccca gatcatatga aacagcatga ctttttcaag
agtgccatgc ccgaaggtta tgtacaggaa agaactatat
ttttcaaaga tgacgggaac tacaagacac gtgctgaagt
caagtttgaa ggtgataccc ttgttaatag aatcgagtta
aaaggtattg attttaaaga agatggaaac attcttggac
acaaattgga atacaactat aactcacaca atgtatacat
catggcagac aaacaaaaga atggaatcaa agttaacttc
aaaattagac acaacattga agatggaagc gttcaactag
cagaccatta tcaacaaaat actccaattg gcgatggccc
tgtcctttta ccagacaacc attacctgtc cacacaatct
gccctttcga aagatcccaa cgaaaagaga gaccacatgg
tccttcttga gtttgtaaca gctgctggga ttacacatgg
catggatgaa ctatacaaat aaatgtccag acttccaatt
gacactaaag tgtccgaaca attactaaaa tctcagggtt
cctggttaaa ttcaggctga gatattattt atatatttat
agattcatta aaattgtatg aataatttat tgatgttatt
gatagaggtt attttcttat taaacaggct acttggagtg
tattcttaat tctatattaa ttacaatttg atttgacttg
ctcaaa.

### REFERENCES

1. Tsien, R. Y. Building and breeding molecules to spy on cells and tumors. FEBS Lett. 579, 927-932 (2005).
2. Zimmer, M. Green fluorescent protein (GFP): applications, structure, and related photophysical behavior. Chem. Rev. 102, 759-781 (2002).
3. Chudakov, D. M., Belousov, V. V., Zaraisky, A. G., Novoselov, V. V., Staroverov, D. B., Zorov, D. B., Lukyanov, S. & Lukyanov, K. A. Kindling fluorescent proteins for precise in vivo photolabeling. Nat. Biotechnol. 21, 191-194 (2003).
4. Ando, R., Mizuno, H. & Miyawaki, A. Regulated fast nucleocytoplasmic shuttling observed by reversible protein highlighting. Science 306, 1370-1373 (2004).
5. Chudakov, D. M., Verkhusha, V. V., Staroverov, D. B., Souslova, E. A., Lukyanov, S. & Lukyanov, K. A. Photoswitchable cyan fluorescent protein for protein tracking. Nat. Biotechnol. 22, 1435-1439 (2004).
6. Ozawa, T., Sako, Y., Sato, M., Kitamura, T. & Umezawa, Y. A genetic approach to identifying mitochondrial proteins. Nat. Biotechnol. 21, 287-293 (2003).
7. Hu, C. D. & Kerppola, T. K. Simultaneous visualization of multiple protein interactions in living cells using multicolor fluorescence complementation analysis. Nat. Biotechnol. 21, 539-545 (2003).
8. Remy, I. & Michnick, S. W. A cDNA library functional screening strategy based on fluorescent protein complementation assays to identify novel components of signaling pathways. Methods 32, 381-388 (2004).
9. Magliery, T. J., Wilson, C. G., Pan, W., Mishler, D., Ghosh, I., Hamilton, A. D. & Regan, L. Detecting protein-protein interactions with a green fluorescent protein fragment reassembly trap: scope and mechanism. J. Am. Chem. Soc. 127, 146-157 (2005).
10. Seeman, N. C. DNA in a material world. Nature 421, 427-431 (2003).
11. Samori, B. & Zuccheri, G. DNA codes for nanoscience. Angew. Chem. Int. Ed. Engl. 44, 1166-1181 (2005).
12. Niemeyer, C. M., Koehler, J. & Wuerdemann, C. DNA-directed assembly of bi-enzymic complexes from in vivo biotinylated NADP(H):FMN oxidoreductase and luciferase. Chem Bio Chem 3, 242-245 (2002).
13. Saghatelian, A., Guckian, K. M., Thayer, D. A. & Ghadiri, M. R. DNA detection and signal amplification via an engineered allosteric enzyme. J. Am. Chem. Soc. 125, 344-345 (2003).
14. Shen, W., Bruist, M. F., Goodman, S. D. & Seeman, N. C. A protein-driven DNA molecule that measures the excess binding energy of proteins that distort DNA. Angew. Chem. Int. Ed. Engl. 43, 4750-4752 (2004).
15. Heyduk, E. & Heyduk. T. Nucleic acid-based fluorescence sensors for detecting proteins. Anal Chem. 77, 1147-1156 (2005).
16. Ormo, M., Cubitt, A. B., Kallio, K., Gross, L. A., Tsien, R. Y & Remington, S. J. Crystal structure of the Aequorea victoria green fluorescent protein. Science 273, 1392-1395 (1996).
17. Reid, B. G. & Flynn, G. C. Chromophore formation in green fluorescent protein. Biochemistry 36, 6786-6791 (1997).
18. Ding, F. & Dokholyan, N. V. Simple yet predictive protein models. Trends Biotechnol. 23 (2005; in press).
19. Jung, G., Wiehler J. & Zumbusch, A. The photophysics of green fluorescent protein: influence of the key amino acids at positions 65, 203, and 222. Biophys. J. 88, 1932-1947 (2005).
20. Hamad-Schifferli, K., Schwartz, J. J., Santos, A. T., Zhang, S. & Jacobson, J. M. Remote electronic control of DNA hybridization through inductive coupling to an attached metal nanocrystal antenna. Nature 415, 152-155 (2002).
21. Evans, T. C. Jr., Benner, J. & Xu, M.-Q. The cyclization and polymerization of bacterially expressed proteins using modified self-splicing inteins. J. Biol. Chem. 274, 18359-18363 (1999).
22. Wang, H. & Chong, S. Visualization of coupled protein folding and binding in bacteria and purification of the heterodimeric complex. Proc. Natl. Acad. Sci. USA 100, 478-483 (2003).
23. Akemann, W., Raj, C. D. & Knopfel, T. Functional characterization of permuted enhanced green fluorescent proteins comprising varying linker peptides. Photochem. Photobiol. 74, 356-363 (2001).
24. Niwa, H., Inouye, S., Hirano, T., Matsuno, T., Kojima, S., Kubota, M., Ohashi, M., & Tsuji, F. I. Chemical nature of the light emitter of the Aequorea green fluorescent protein. Proc. Natl. Acad. Sci. USA 93, 13617-13622 (1996).
25. Coussaert, T., Volkel, A. R., Noolandi, J. & Gast, A. P. Streptavidin tetramerization and 2D crystallization: a mean-field approach. Biophys. J. 80, 2004-2010 (2001).
26. Freitag, S., Le Trong, I., Klumb, L., Stayton, P. S. & Stenkamp, R. E. Structural studies of the streptavidin binding loop. Protein Sci. 6, 1157-1166 (1997).
27. Gothelf, K. V. & Brown, R. S. A modular approach to DNA-programmed self-assembly of macromolecular nanostructures. Chem. Eur. J. 11, 1062-1069 (2005).
28. Niemeyer, C. M., Sano, T., Smith, C. L. & Cantor, C. R. Oligonucleotide-directed self-assembly of proteins: semisynthetic DNA-streptavidin hybrid molecules as connectors for the generation of macroscopic arrays and the construction of supramolecular bioconjugates. Nucleic Acids Res. 22, 5530-5539 (1994).
29. Dickson, R. M., Cubitt, A. B., Tsien, R. Y. & Moerner, W. E. On/off blinking and switching behaviour of single molecules of green fluorescent protein. Nature 388, 355-358 (1997).
30. Stains, C. I., Porter, J. R., Ooi, A. T. Segal, D. J. & Ghosh, I. DNA sequence-enabled reassembly of the green fluorescent protein. J. Am. Chem. Soc. 127, 10782-10783 (2005).
31. Berendsen, H. J. C., J. P. M. Postma, W. F. van Gunsteren, A. Di Nola, and J. R. Haak (1984) Molecular dynamics with coupling to an external bath. J. Chem. Phys. 81:3684-3690.
32. Shyu, Y. J., Liu, H., Deng, X., Hu, C. D. Identification of new fluorescent protein fragments for biomolecular fluorescence complementation analysis under physiological conditions. BioTechniques, 40, 61-66 (2006).

All references described herein are incorporated herein by reference in their entirety.

We claim:

1. A method for the detection of diseases or disorders in an individual comprising:
a. obtaining a test biological sample from an individual;
b. isolating DNA or RNA from the biological sample;
c. contacting the DNA or RNA with a split-fluorescent polypeptide molecule, wherein the split-fluorescent polypeptide fragments are conjugated to nucleic acid binding motifs, and wherein a least one the nucleic acid binding motif is specific for a particular nucleic acid that is associated with a disease or disorder; and
d. detecting a change in signal from the detectable protein, wherein the change in signal is indicative of the presence of a disease or disorder.

2. A method for the detection of diseases or disorders in an individual comprising:
a. obtaining a test biological sample from an individual;
b. isolating an non-nucleic acid analyte from the biological sample;
c. contacting the non-nucleic acid analyte with a split-fluorescent polypeptide molecule, wherein the split-fluorescent polypeptide fragments are conjugated to binding motif for the non-nucleic analyte, and wherein a least one the analyte binding motif is specific for a particular nucleic acid that is associated with a disease or disorder; and
d. detecting a change in signal from the detectable protein, wherein the change in signal is indicative of the presence of a disease or disorder.

3. The method of claims 1 and 2, wherein the split-fluorescent polypeptide comprises:
a. a first fragment of an EGFR peptide comprising amino acid 1 to approximately amino acid 158; and
b. a second fragment of an EGFR peptide comprising approximately amino acid 159 to amino acid 239; and
c. a cleavage peptide located between the first and the second EGFR fragments.

4. The method of claims 1 and 2, wherein the disease is a pathogen.

5. The method of claims 1 and 2, wherein the pathogen is selected from a group comprising; virus, influenza, bacteria, fungus, parasite or yeast.

6. The method of claim 4, wherein the pathogen is a virus.

7. The method of claims 1 and 2, wherein the disease is a genetic disposition to a disease.

8. A preparation of inclusion bodies comprising a split-fluorescent polypeptide, wherein said split-fluorescent polypeptide comprises:
a. a first fragment of an EGFR peptide comprising amino acid 1 to approximately amino acid 158; and
b. a second fragment of an EGFR peptide comprising approximately amino acid 159 to amino acid 239; and
c. a cleavage peptide located between the first and the second EGFR fragments.

9. A split-polypeptide protein fragment molecule, comprising at least two polypeptide fragments of a detectable protein, wherein the fragments: (a) are in an activated form (b) are not active by themselves; (c) further comprise a nucleic acid binding motif; and (d) rapidly complement to reconstitute the active protein in real time in the presence of a target nucleic acid.

10. The split-polypeptide protein fragment molecule of claim 7, wherein the target nucleic acid is selected from a group comprising: DNA, RNA, PNA and analogues thereof.

11. A split-polypeptide protein fragment molecule, comprising at least two polypeptide fragments of a detectable protein, wherein the fragments: (a) are in an activated form (b) are not active by themselves; (c) further comprise a binding motif for a non-nucleic acid analyte; and (d) rapidly complement to reconstitute the active protein in real time in the presence of a target analyte molecule.

12. The split-polypeptide protein fragment molecule of claim 9, wherein the target analyte molecule is a biomolecule, organic molecule or inorganic molecule.

13. The split-polypeptide protein fragment molecule of claims 9 and 11, wherein the detectable protein is a fluorescent protein.

14. The split-polypeptide protein fragment molecule of claims 9 and 11, wherein the fluorescent protein is selected from a group consisting of green fluorescent protein (GFP), GFP-like fluorescent proteins, (GFP-like); enhanced green fluorescent protein (EGFP); yellow fluorescent protein (YFP); enhanced yellow fluorescent protein (EYFP); blue fluorescent protein (BFP); enhanced blue fluorescent protein (EBFP); cyan fluorescent protein (CFP); enhanced cyan fluorescent protein (ECFP); and red fluorescent protein (dsRED) and variants thereof.

15. The split-polypeptide protein fragment molecule of claims 9 and 11, wherein the molecule is a split-fluorescent protein molecule, and wherein one polypeptide fragment comprises a mature chromophores of a fluorescent protein and where the split-fluorescent fragments of the molecule: (a) together contain the full complement of beta-strands in the chromophore-shielding barrel of a fluorescent protein; (b) are not fluorescent by themselves; (c) further comprise a nucleic acid binding motif; and (d) rapidly complement to reconstitute the fluorescent protein and fluorescent phenotype in real time in the presence of target nucleic acid or target analyte molecule.

16. The split-polypeptide protein fragment molecule of claim 9, wherein the fluorescent protein is EGFP.

17. The split-polypeptide protein fragment molecule of claim 9, wherein the nucleic acid binding motif is selected from a group comprising DNA, RNA, PNA, LNA DNA-binding proteins or peptides; RNA-binding proteins or peptides.

18. The split-polypeptide protein fragment molecule of claim 9, wherein the nucleic acid binding motif on one fragment is of the same type as the nucleic acid binding fragment on the other fragment.

19. The split-polypeptide protein fragment molecule of claim 9, wherein the nucleic acid binding motif on one fragment is of a different type as the nucleic acid binding fragment on the other fragment.

20. A method for the real time detection of changes in nucleic acid hybridization, the method comprising: (a) detecting a baseline signal of the molecule as described in claim 2, wherein the nucleic acid binding motif on one fragment is bound to the nucleic acid binding motif on the second fragment with a nucleic acid in a biological sample; (b) altering the assay conditions such that there may be an alteration in the binding of the two fragments in the sample; and (c) immediately detecting a change in the fluorescent signal from the biological sample, wherein a reduction in signal is indicative that the alteration in the assay conditions decreased the affinity of the separate polypeptide fragments for its original nucleic acid target.

21. The method of claim 20, wherein the nucleic acid binding motif on one fragment is the same type of nucleic acid binding motif on the second fragment.

22. The method of claim 20, wherein the nucleic acid binding motif on one fragment is a different type of nucleic acid binding motif on the second fragment.

23. A method for the production of activated split-polypeptide protein fragments comprising:
a. expressing a nucleic acid sequence encoding a first polypeptide fragment and at least one other polypeptide fragment, wherein the two polypeptide fragments combine in the presence of a target nucleic acid or target non-nucleic acid analyte to form a detectable protein in its active state, wherein the polypeptide fragments are in an activated and conformationally correct form when compared to an active wild type protein; and
b. harvesting said polypeptide fragments to obtain two separate protein fragments in a conformationally correct and activated state.

24. The method of claim 21, wherein the nucleic acid sequence encoding a first polypeptide fragment and at least one other polypeptide fragment are encoded as one nucleic acid sequence, wherein the nucleic acid sequence encodes a splittable site between first polypeptide fragment and the other polypeptide fragments, wherein the first polypeptide fragment and other polypeptide fragments can be separated and are in the activated and conformationally correct form when compared to an active wild type protein.

25. The method of claim 24, wherein the splittable site enables separation of the first polypeptide fragment from the other polypeptide fragments by cleavage means selected from a group consisting of; enzymatic cleavage; chemical cleavage; photocleavage; wavelength cleavage; heat cleavage; acid cleavage.

26. The method of claim 23, comprising:
a. expressing a nucleic acid sequence encoding a first polypeptide fragment and at least one other polypeptide fragment in a microbial host cell to form inclusion bodies, wherein the inclusion bodies comprise said polypeptide fragments; and
b. lysing the host cell, harvesting the inclusion bodies and resolubilizing and refolding the polypeptide fragments contained in said inclusion bodies of step (a) to obtain the first polypeptide fragment and at least one other polypeptide fragment in their activated conformation.

27. The method of claim 26, further comprising enzymatically or chemically splitting the polypeptide comprising the first and at least one other polypeptide fragment, to obtain the first and at least one other polypeptide fragment in their activated state.

28. The method of claim 26, further comprising harvesting the polypeptide fragments from the soluble fraction of said host cell to obtain the first polypeptide and at least one other polypeptide fragment in their activated conformation

29. The method of claim 23, wherein the detectable protein is an enzyme.

30. The method of claim 25, wherein the enzyme has chromogenic activity.

31. The method of claim 23, wherein the detectable protein is a fluorescent protein.

32. The method of claim 23, wherein the first polypeptide fragment of a fluorescent protein comprises a mature preformed chromophores that is primed for fluorescence.

33. The method of claim 31, wherein the fluorescent protein is selected from a group comprising; green fluorescent protein (GFP); enhanced green fluorescent protein (EGFP); yellow fluorescent protein (YFP); enhanced yellow fluorescent protein (EYFP); blue fluorescent protein (BFP); enhanced blue fluorescent protein (EBFP); cyan fluorescent protein (CFP); enhanced cyan fluorescent protein (ECFP); red fluorescent protein (dsRED); and variants thereof.

34. The method of claim 31, wherein the fluorescent protein is the EGFP fluorescent protein.

35. The method of claim 34, wherein the EGFP fluorescent protein comprises a first polypeptide fragment protein comprising of amino acid 1 to approximately amino acid 158, and wherein a second polypeptide fragment of the EGFP fluorescent protein is approximately amino acid 159 to amino acid 239.

36. The method of claim 23, wherein the first polypeptide fragment further comprises a C-terminal cysteine and the second polypeptide fragment further comprises an N-terminal cysteine.

37. The method of claim 23, further comprising biotinylating the first and at least one other polypeptide fragments with a sulfhydryl-reactive reagent.

38. The method of claim 37, wherein the sulfhydryl-reactive reagent is biotin-HPDP.

39. The method of claim 23, wherein the first and at least another polypeptide fragments are further conjugated to streptavidin-conjugated oligonucleotide.

40. The method of claim 39, wherein the oligonucleotide is selected from a group comprising DNA, RNA, PNA, LNA and analogues thereof.

41. The method of claim 23, wherein nucleic acid encoding the first and at least one polypeptide fragment further encodes a nucleic acid binding moiety.

42. The method of claim 41, wherein the nucleic acid binding moiety is a nucleic acid.

43. The method of claim 42, wherein the nucleic acid binding moiety is conjugated to the first and at least one other polypeptide fragment.

44. The method of claim 42, wherein the nucleic acid binding moiety is selected from a group comprising; DNA-binding proteins; DNA-binding peptides; RNA-binding proteins; RNA-binding peptides.

45. A kit comprising;
a. a first and at least one other activated split-polypeptide fragment, wherein each split-polypeptide fragment comprises a nucleic acid binding domain or binding motif for non-nucleic acid analyte;
b. reagents and instructions for complementation and signal detection;

46. A kit comprising;
a. a first and at least one other activated split-polypeptide fragment;
b. reagents and instructions for the attachment of the users own nucleic acid binding motif of interest or binding motif for non-nucleic acid analyte;
c. reagents and instructions for complementation and signal detection;

47. The kit of claims 45 and 46, wherein the first and second activated split-polypeptide fragments reconstitute to form a detectable protein.

48. The kit of claim 47, wherein the detectable protein is selected from a list comprising; β-lactamase; DFHR; luciferase; fluorescent protein.

49. The kit of claim 47, wherein the detectable protein is an antigen.

50. The kits of claims 45 and 46 further comprising reagents and instructions for amplification of the target nucleic acid of the sample.

51. The method of claims 1 and 2, wherein the change is a reduction in signal.

52. The method of claims 1 and 2, wherein the change is an increase in signal.
